(19) United States
(12) Patent Application Publication
(10) Pub. No.: US 2021/0298565 A1
(43) Pub. Date: Sep. 30, 2021

(54) FLEXIBLE TUBE FOR ENDOSCOPE, ENDOSCOPIC MEDICAL DEVICE, AND METHODS FOR PRODUCING THE SAME

(71) Applicant: FUJIFILM Corporation, Tokyo (JP)

(72) Inventors: Yoshihiro Nakai, Ashigarakami-gun (JP); Shinya Abe, Ashigarakami-gun (JP)

(73) Assignee: FUJIFILM Corporation, Tokyo (JP)

(21) Appl. No.: 17/344,359

(22) Filed: Jun. 10, 2021

Related U.S. Application Data
(63) Continuation of application No. PCT/JP2020/000877, filed on Jan. 14, 2020.

(30) Foreign Application Priority Data
Jan. 16, 2019 (JP) 2019-005385

(51) Int. Cl. A61B 1/005; A61B 1/00; B05D 7/14; B05D 7/00

(52) U.S. Cl. CPC A61B 1/005; A61B 1/0011; B05D 7/146; B05D 2202/15; B05D 2505/00; B05D 2350/60; B05D 7/52

(57) ABSTRACT

Provided are a flexible tube for an endoscope, the flexible tube having a flexible tube base made of metal, a resin cover layer that covers an outer periphery of the flexible tube base, and a primer layer that includes at least one compound represented by general formula (1) or (2) and that is disposed between the flexible tube base and the resin cover layer, in which the resin cover layer includes at least one selected from the group consisting of polyamides, polyesters, polyurethanes, and polyolefins on a side of the resin cover layer in contact with the primer layer, an endoscopic medical device including the flexible tube for an endoscope; a method for producing the flexible tube for an endoscope; and a method for producing the endoscopic medical device.

R¹ₘ-M-(OR²)ₙ₋ₘ General formula (1):

O-[M-(OR²)ₙ₋₁]₂ General formula (2):
M represents, for example, Al, Ti, or Zr.
R¹ and R² each represent a hydrogen atom or a specific group.
m is an integer of 0 to 3, n is a valence of M, and n>m is satisfied.

### CROSS-REFERENCE TO RELATED APPLICATIONS

This application is a Continuation of PCT International Application No. PCT/JP2020/000877 filed on Jan. 14, 2020, which claims priority under 35 U.S.C. § 119 (a) to Japanese Patent Application No. 2019-005385 filed in Japan on Jan. 16, 2019. Each of the above applications is hereby expressly incorporated by reference, in its entirety, into the present application.

### BACKGROUND OF THE INVENTION

### 1. Field of the Invention

The present invention relates to a flexible tube for an endoscope, an endoscopic medical device, and methods for producing the same.

### 2. Description of the Related Art

Endoscopes are medical devices for examining the inside of the body cavity, the inside of the digestive tract, the esophagus, or the like of a patient. Since endoscopes are inserted and used in the body, it is desirable to provide endoscopes that do not damage organs or cause pain or discomfort to a patient. In view of such a requirement, a spiral tube formed by winding a soft, bendable metal strip in a spiral form is adopted as a flexible tube that forms an insertion section (structural section to be inserted into a body cavity) of an endoscope. Furthermore, the periphery of the spiral tube is covered with a flexible resin, and this resin cover layer is covered with a topcoat layer, as needed, so that the spiral tube does not cause stimulation or damage to an inner surface of, for example, the esophagus, digestive tract, or body cavity.

The resin cover layer can be formed by, for example, extrusion-molding a resin on an outer peripheral surface of a flexible tube base that is formed by covering a spiral tube with a tubular mesh. In this case, it is preferable to make the distal end side soft so as to easily insert the flexible tube into the body and to make the proximal end side hard so as to easily perform the operation. In consideration of this point, it has been proposed that a two-layer structure having an inner layer and an outer layer that have different degrees of hardness is adopted as the resin cover layer, and a ratio of the thickness of the inner layer to the thickness of the outer layer is changed in the axial direction of the flexible tube.

To improve operability, durability, and the like of an endoscope, it is important to enhance adhesiveness between a flexible tube base and a resin cover layer that covers the flexible tube base. If this adhesiveness is insufficient, when a flexible tube is inserted into the body, for example, a crease, floating, tearing, or separation is easily caused on the resin cover layer by bending of the flexible tube. In addition, when the flexible tube is inserted and then rotated in the inserted state, twisting of the resin cover layer tends to occur. If such a crease, floating, tearing, separation, or twisting is caused in the resin cover layer, for example, the surface of the flexible tube inserted in the body catches the peripheral tissues, which may cause a pain to a subject. It is known that the adhesiveness between the flexible tube base and the resin cover layer is decreased by, for example, sterilization treatment of the flexible tube.

It is known that a primer layer is disposed between the flexible tube base and the resin cover layer to increase the adhesiveness. For example, JP2011-212338A discloses that a primer is applied to a surface of a metal core (flexible tube base) and an outer coating layer is then formed so as to cover the primer and that a silane coupling agent, a titanate coupling agent, a zirconate coupling agent, or the like can be used as the primer.

### SUMMARY OF THE INVENTION

An object of the present invention is to provide a flexible tube for an endoscope, the flexible tube being capable of sufficiently maintaining adhesiveness between a flexible tube base and a resin cover layer that covers the flexible tube base even when a bending operation is repeated and being less likely to undergo a decrease in the adhesiveness between the flexible tube base and the resin cover layer even when the flexible tube is subjected to strong sterilization treatment using ethylene oxide gas and to provide an endoscopic medical device that includes the flexible tube for an endoscope. Another object of the present invention is to provide a method for producing the flexible tube for an endoscope and a method for producing the endoscopic medical device.

In view of the problems described above, the inventors of the present invention have conducted extensive studies on formation of a resin cover layer in a flexible tube for an endoscope. As a result, the inventors have found that the above objects can be achieved by forming a primer layer that includes a compound having a specific structure, such as a metal alkoxide, on a surface of a flexible tube base formed of a metal material, and using a specific resin as a constituent material of a resin cover layer that is in contact with the primer layer. The inventors have further conducted studies on the basis of these findings and completed the present invention.

The objects of the present invention have been achieved by the following means.

[1]

A flexible tube for an endoscope, the flexible tube having a flexible tube base containing metal as a constituent material; a resin cover layer that covers an outer periphery of the flexible tube base; and a primer layer that includes at least one compound represented by general formula (1) or (2) and that is disposed between the flexible tube base and the resin cover layer, in which the resin cover layer includes at least one compound selected from the group consisting of polyamides, polyesters, polyurethanes, and polyolefins at least on a side of the resin cover layer in contact with the primer layer.

R¹ₘ-M-(OR²)ₙ₋ₘ General formula (1):

O-[M-(OR²)ₙ₋₁]₂ General formula (2):

In the formulae, M represents Al, B, Ba, Bi, Ca, Ga, Ge, Hf, In, La, Mg, Nb, P, Sr, Sn, Ta, Ti, V, Y, or Zr.

R¹ represents a hydrogen atom, an alkyl group, a cycloalkyl group, an acyl group, an aryl group, or an unsaturated aliphatic group.

R² represents a hydrogen atom, an alkyl group, a cycloalkyl group, an acyl group, an alkenyl group, an aryl group, a phosphonate group, or —SO₂R¹ where R^{s} represents a substituent.

m is an integer of 0 to 3, n is a valence of M, and n>m is satisfied.

[2]

The flexible tube for an endoscope according to [1], in which the M is Ti.

[3]

The flexible tube for an endoscope according to [2], in which the compound represented by general formula (1) or (2) includes at least one atom selected from the group consisting of N, P, and S.

[4]

The flexible tube for an endoscope according to [1], in which the M is Al.

[5]

The flexible tube for an endoscope according to [4], in which at least one of OR² in general formulae (1) or (2) has an acetonato structure.

[6]

The flexible tube for an endoscope according to [4] or [5], in which at least one of OR² in general formulae (1) or (2) has an acetato structure.

[7]

The flexible tube for an endoscope according to [1], in which the M is Zr.

[8]

The flexible tube for an endoscope according to [7], in which at least one of OR² in general formulae (1) or (2) has an acetonato structure.

[9]

The flexible tube for an endoscope according to [7] or [8], in which at least one of OR² in general formulae (1) or (2) has an acetato structure.

[10]

The flexible tube for an endoscope according to any of [7] to [9], in which at least one of OR² in general formulae (1) or (2) has a lactato structure.

[11]

The flexible tube for an endoscope according to any of [1] to [10], in which the metal that constitutes the flexible tube base is stainless steel.

[12]

The flexible tube for an endoscope according to any of [1] to [11], in which the metal that constitutes the flexible tube base has a passivation film on a surface thereof.

[13]

The flexible tube for an endoscope according to any of [1] to [12], in which the resin cover layer has a single-layer structure or a multilayer structure and includes at least one compound selected from the group consisting of polyamides, polyesters, polyurethanes, and polyolefins in a layer in contact with the primer layer.

[14]

The flexible tube for an endoscope according to any of [1] to [13], in which the resin cover layer has a two-layer structure, and a ratio of a thickness of an inner layer to a thickness of an outer layer of the two-layer structure changes in a gradient manner in an axial direction of the flexible tube base.

[15]

The flexible tube for an endoscope according to [14], in which the ratio of the thickness of the inner layer to the thickness of the outer layer is inner layer:outer layer=95:5 to 60:40 at one end of the flexible tube for an endoscope and is inner layer:outer layer=5:95 to 40:60 at the other end.

[16]

An endoscopic medical device having the flexible tube for an endoscope according to any of [1] to [15].

[17]

A method for producing a flexible tube for an endoscope, the method including: a step of forming, on at least an outer periphery of a flexible tube base that contains metal as a constituent material, a primer layer that includes at least one compound represented by general formula (1) or (2); and

a step of forming a resin cover layer, the step including covering, with a resin that includes at least one compound selected from the group consisting of polyamides, polyesters, polyurethanes, and polyolefins, the primer layer formed on the outer periphery of the flexible tube base so as to be in contact with the primer layer.

R¹ₘ-M-(OR²)ₙ₋ₘ General formula (1):

O-[M-(OR²)ₙ₋₁]₂ General formula (2):

In the formulae, M represents Al, B, Ba, Bi, Ca, Ga, Ge, Hf, In, La, Mg, Nb, P, Sr, Sn, Ta, Ti, V, Y, or Zr.

R¹ represents a hydrogen atom, an alkyl group, a cycloalkyl group, an acyl group, an aryl group, or an unsaturated aliphatic group.

R² represents a hydrogen atom, an alkyl group, a cycloalkyl group, an acyl group, an alkenyl group, an aryl group, a phosphonate group, or —SO₂R^{s} where R^{s} represents a substituent.

m is an integer of 0 to 3, n is a valence of M, and n>m is satisfied.

[18]

The method for producing a flexible tube for an endoscope according to [17], in which the resin cover layer has a two-layer structure, at least an inner layer of the two-layer structure includes at least one compound selected from the group consisting of polyamides, polyesters, polyurethanes, and polyolefins, and a ratio of a thickness of the inner layer to a thickness of an outer layer of the two-layer structure changes in a gradient manner in an axial direction of the flexible tube base.

[19]

A method for producing an endoscopic medical device, including, a step of producing a flexible tube for an endoscope by the method for producing a flexible tube for an endoscope according to [17] or [18]; and a step of incorporating the produced flexible tube for an endoscope into an insertion section of an endoscopic medical device.

[20]

A method for producing an endoscopic medical device, including incorporating the flexible tube for an endoscope according to any of [1] to [15] into an insertion section of an endoscopic medical device.

In the present specification, when a plurality of substituents, linking groups, or the like (hereinafter referred to as substituents or the like) represented by specific symbols are present or a plurality of substituents or the like are defined simultaneously or alternatively, the substituents or the like may be the same or different from each other. In addition, even if not specifically stated, when a plurality of substituents or the like are adjacent to each other, they may be linked or fused to each other to form a ring.

In the present specification, if it is not explicitly specified whether a substituent is substituted or unsubstituted (the same applies to a linking group), it is meant that the group may have any substituent within the range in which the desired effect is achieved. This also applies if it is not explicitly specified whether a compound is substituted or unsubstituted.

In the present specification, when the number of carbon atoms of a group is specified, the number of carbon atoms means the number of carbon atoms of the whole group. That is, in the case of a form where the group further has a substituent, the number of carbon atoms means the number of carbon atoms of the whole that includes this substituent.

The flexible tube for an endoscope according to the present invention is capable of sufficiently maintaining adhesiveness between a flexible tube base and a resin cover layer that covers the flexible tube base even when a bending operation is repeated, and is less likely to undergo a decrease in the adhesiveness between the flexible tube base and the resin cover layer even when the flexible tube is subjected to sterilization treatment.

According to the endoscopic medical device according to the present invention, a flexible tube which is a structural section to be inserted into a body is capable of sufficiently maintaining adhesiveness between a flexible tube base and a resin cover layer that covers the flexible tube base even when a bending operation is repeated, and is less likely to undergo a decrease in the adhesiveness between the flexible tube base and the resin cover layer even when the flexible tube is subjected to sterilization treatment. Therefore, the endoscopic medical device according to the present invention has good durability, and the load on a subject during use of the endoscopic medical device can be further reduced.

The method for producing a flexible tube for an endoscope according to the present invention can provide a flexible tube for an endoscope, the flexible tube being capable of sufficiently maintaining adhesiveness between a flexible tube base and a resin cover layer that covers the flexible tube base even when a bending operation is repeated and being less likely to undergo a decrease in the adhesiveness between the flexible tube base and the resin cover layer even when the flexible tube is subjected to sterilization treatment.

According to the method for producing an endoscopic medical device according to the present invention, a flexible tube that forms this device can have such properties that adhesiveness between a flexible tube base and a resin cover layer that covers the flexible tube base can be sufficiently maintained even when a bending operation is repeated and that the adhesiveness between the flexible tube base and the resin cover layer is less likely to decrease even when the flexible tube is subjected to sterilization treatment. Therefore, the method for producing an endoscopic medical device according to the present invention can provide an endoscopic medical device which has good durability and in which the load on a subject during use is further reduced.

### BRIEF DESCRIPTION OF THE DRAWINGS

FIG. 1 is an external view illustrating a configuration of an electronic endoscope according to an embodiment;

FIG. 2 is a partial sectional view illustrating a configuration of a flexible tube for an endoscope according to an embodiment;

FIG. 3 is a block diagram illustrating a configuration of an apparatus for producing a flexible tube for an endoscope according to an embodiment; and

FIG. 4 is a sectional view taken along line B-B in FIG. 3.

### DESCRIPTION OF THE PREFERRED EMBODIMENTS

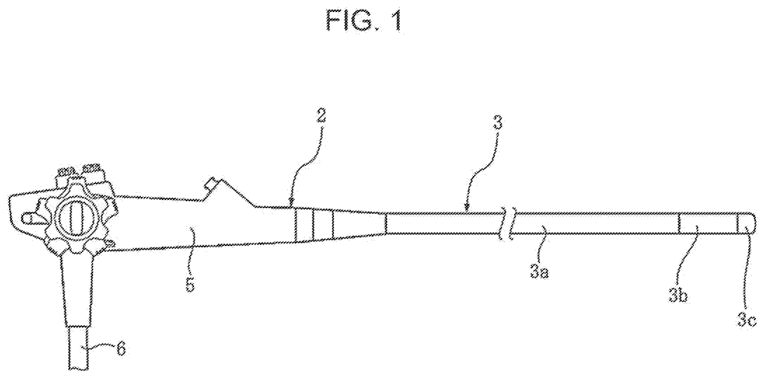

An electronic endoscope will now be described as an example of an endoscopic medical device according to a preferred embodiment of the present invention. An electronic endoscope includes a flexible tube for an endoscope (hereinafter, a flexible tube for an endoscope may be simply referred to as a “flexible tube”), the flexible tube being incorporated in the electronic endoscope, and is used as a medical device for, for example, examining the inside of the body by inserting the flexible tube into the inside of the body cavity, the inside of the digestive tract, the esophagus, or the like. In the example illustrated in FIG. 1, an electronic endoscope 2 includes an insertion section 3 to be inserted into a body, a main body operating section 5 that is connected to a proximal end portion of the insertion section 3, and a universal cord 6 to be connected to a processor device or a light source device. The insertion section 3 includes a flexible tube 3a connected to the main body operating section 5, an angle portion 3b connected to the flexible tube 3a, and a tip portion 3c which is connected to the distal end of the angle portion 3b and in which an imaging device (not shown) for imaging the inside of the body is installed. The flexible tube 3a that accounts for a large portion of the length of the insertion section 3 has flexibility across substantially the entire length thereof and is configured so that, in particular, a portion to be inserted into the inside of a body cavity or the like has higher flexibility.

### Flexible Tube Base

The flexible tube has, as an innermost layer, a flexible tube base containing metal as a constituent material.

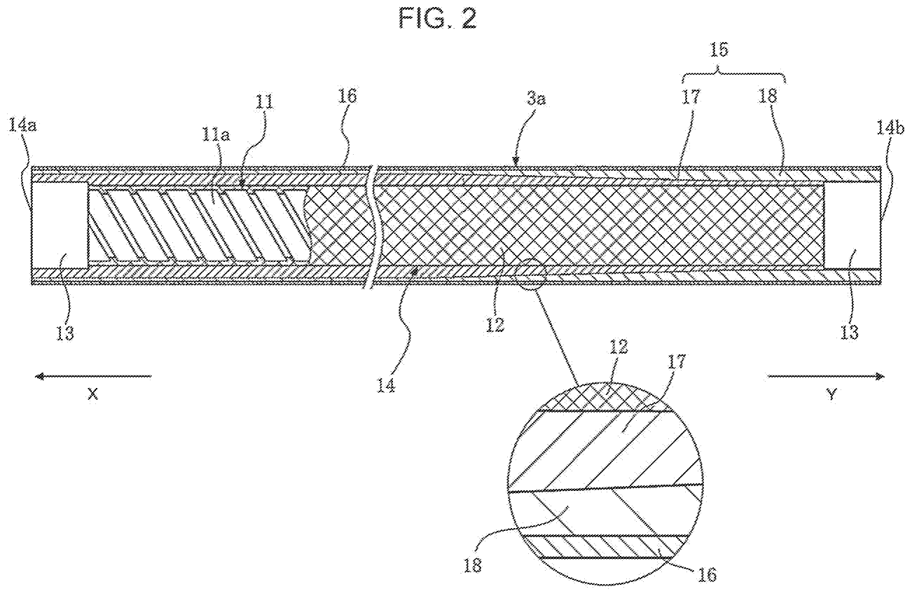

As illustrated in FIG. 2, a flexible tube base 14 preferably has a form in which a spiral tube 11 that is formed, on the innermost side, by winding a metal strip 11a in a spiral form is covered with a tubular mesh 12 obtained by braiding metal wires, and caps 13 are fitted in both ends of the resulting product. The metal constituting the flexible tube base 14 preferably has a surface that has been subjected to passivation treatment in order to prevent corrosion. That is, the flexible tube base 14 preferably has a passivation film on an outer periphery thereof. This passivation treatment can be performed by an ordinary method. A passivation film can be formed on a surface of metal by, for example, immersing the metal in a solution including a strong oxidizing agent such as nitric acid, heating the metal in air (oxygen) or water (water vapor), or anodizing the metal in a solution including an oxidizing agent.

The metal that constitutes the flexible tube base 14 is preferably stainless steel. The surface of stainless steel is usually in a state in which chromium and oxygen are bound to each other to form a passivation film. However, even in the case where stainless steel is used as the constituent material of the flexible tube base 14, the stainless steel is preferably subjected to the passivation treatment described above so that a more uniform passivation film is more reliably formed over the entire surface of the stainless steel.

### Primer Layer

In the present invention, a primer layer (not shown) is disposed on an outer periphery of the flexible tube base. By disposing this primer layer, it is possible to effectively enhance adhesiveness between the flexible tube base and a resin cover layer described below and provided to cover the outer periphery of the flexible tube base. In the present invention, this primer layer includes at least one compound represented by general formula (1) or (2) below. That is, the primer layer in the present invention includes the following forms (i) to (iii):

(i) a form that includes at least one compound represented by general formula (1) and that does not include a compound represented by general formula (2);
(ii) a form that does not include a compound represented by general formula (1) and that includes at least one compound represented by general formula (2); and
(iii) a form that includes at least one compound represented by general formula (1) and that includes at least one compound represented by general formula (2).

R¹ₘ-M-(OR²)ₙ₋ₘ General formula (1):

O-[M-(OR²)ₙ₋₁]₂ General formula (2):

In general formulae (1) and (2), M represents Al, B, Ba, Bi, Ca, Ga, Ge, Hf, In, La, Mg, Nb, P, Sr, Sn. Ta, Ti, V, Y, or Zr. M is preferably Ti, Al, or Zr.

R¹ represents a hydrogen atom, an alkyl group, a cycloalkyl group, an acyl group, an aryl group, or an unsaturated aliphatic group.

The alkyl group that may be employed as R¹ includes linear alkyl groups, branched alkyl groups, and aralkyl groups. The number of carbon atoms of this alkyl group is preferably an integer of 1 to 20, more preferably 1 to 15, still more preferably 1 to 10, and particularly preferably 1 to 8. In the case of an aralkyl group, the number of carbon atoms is preferably 7 to 30. Preferred specific examples of this alkyl group include methyl, ethyl, propyl, isopropyl, n-butyl, sec-butyl, isobutyl, tert-butyl, n-pentyl, n-hexyl, n-heptyl, n-octyl, n-decyl, n-tridecyl, n-octadecyl, benzyl, and phenethyl.

The number of carbon atoms of the cycloalkyl group that may be employed as R¹ is preferably an integer of 3 to 20, more preferably 3 to 15, still more preferably 3 to 10, and particularly preferably 3 to 8. Preferred specific examples of this cycloalkyl group include cyclopropyl, cyclopentyl, and cyclohexyl.

The number of carbon atoms of the acyl group that may be employed as R¹ is preferably an integer of 2 to 40, more preferably 2 to 30, still more preferably 2 to 20, and particularly preferably 2 to 18.

The number of carbon atoms of the aryl group that may be employed as R¹ is preferably an integer of 6 to 20, more preferably 6 to 15, still more preferably 6 to 12, and particularly preferably 6 to 10. Preferred specific examples of this aryl group include phenyl and naphthyl. The aryl group is more preferably phenyl.

The number of carbon-carbon unsaturated bonds of the unsaturated aliphatic group that may be employed as R¹ is preferably 1 to 5, more preferably 1 to 3, still more preferably 1 or 2, and particularly preferably 1. The unsaturated aliphatic group may include a heteroatom. It is also preferable that the unsaturated aliphatic group be a hydrocarbon group. When the unsaturated aliphatic group is a hydrocarbon group, the number of carbon atoms is preferably 2 to 20, more preferably 2 to 15, still more preferably 2 to 10, and particularly preferably 2 to 8. The unsaturated aliphatic group is more preferably an alkenyl group or an alkynyl group.

R¹ is preferably a hydrogen atom, an alkyl group, a cycloalkyl group, or an aryl group and more preferably an alkyl group or a cycloalkyl group.

When the compound represented by general formula (1) has two or more R¹, two R¹ may be linked to each other to form a ring.

R² represents a hydrogen atom, an alkyl group, a cycloalkyl group, an acyl group, an alkenyl group, an aryl group, a phosphonate group (phosphonic acid group), or —SO₂R^{s}. R^{s} represents a substituent.

The alkyl group, the cycloalkyl group, the acyl group, and the aryl group that may be employed as R² respectively have the same definition as in the alkyl group, the cycloalkyl group, the acyl group, and the aryl group that may be employed as R¹, and preferred forms thereof are also the same as those of the alkyl group, the cycloalkyl group, the acyl group, and the aryl group that may be employed as R¹. It is also preferable that the alkyl group that may be employed as R² have an amino group as a substituent.

The alkenyl group that may be employed as R² includes linear alkenyl groups and branched alkenyl groups. The number of carbon atoms of this alkenyl group is preferably an integer of 2 to 18, more preferably 2 to 7, and still more preferably 2 to 5. Preferred specific examples of this alkenyl group include vinyl, allyl, butenyl, pentenyl, and hexenyl. This alkenyl group is preferably a substituted alkenyl group.

The phosphonate group that may be employed as R² is a group represented by —P(═O)(—OR^{P1})OR^{P2}. R^{P1} and R^{P2} each represent a hydrogen atom or a substituent, and this substituent is preferably an alkyl group or a phosphonate group. The alkyl group that may be employed as R^{P1} and R^{P2} has the same definition as in the alkyl group that may be employed as R¹, and preferred forms of the alkyl group are also the same as those of the alkyl group that may be employed as R¹. The phosphonate group that may be employed as R^{P1} and R^{P2} has the same definition as in the phosphonate group that may be employed as R², and preferred forms thereof are also the same as those of the phosphonate group that may be employed as R². When R^{P1} or R^{P2} is a phosphonate group, R^{P1} and R^{P2} that form this phosphonate group are preferably alkyl groups.

In the phosphonate group that may be employed as R², R^{P1} and R^{P2} are each preferably an alkyl group, or R^{P1} is preferably a hydrogen atom and R^{P2} is preferably a phosphonate group.

Since a phosphonate group and a phosphite group (phosphorous acid group) are tautomers, the term phosphonate group in the present invention is meant to include a phosphite group.

In —SO₂R^{s} that may be employed as R², the substituent R^{S} is preferably an alkyl group or an aryl group. Examples of preferred forms of the alkyl group and the aryl group that may be employed as R^{S} include preferred forms of the alkyl group and the aryl group, respectively, that may be employed as R¹. In particular, R^{S} is preferably phenyl having an alkyl group as a substituent. Preferred forms of this alkyl group are the same as the preferred forms of the alkyl group that may be employed as R¹.

When the compound represented by general formula (1) or the compound represented by general formula (2) has two or more R², two R² may be linked to each other to form a ring.

m is an integer of 0 to 3, and n is a valence of M. In addition, n>m is satisfied, and m is preferably 0 or 1 and more preferably 0.

When M is Ti, the compound represented by general formula (1) or (2) preferably includes at least one atom selected from the group consisting of N, P, and S. When the compound represented by general formula (1) or (2) has N, the compound preferably has this N as an amino group.

When the compound represented by general formula (1) or (2) has P, the compound preferably has this P as a phosphate group (phosphoric acid group) or a phosphonate group (phosphonic acid group).

When the compound represented by general formula (1) or (2) has S, the compound preferably has this S as a sulfonyl group (—SO₂—).

Ti is usually tetravalent.

When M is Al, at least one of OR² in general formula (1) or (2) above preferably has an acetonato structure. This acetonato structure means a structure obtained by removing one hydrogen ion from acetone or a compound having a structure of acetone with a substituent and coordinating the resulting structure to M. The coordinating atom that coordinates to M is usually an oxygen atom. This acetonato structure is preferably a structure which has an acetylacetone structure “CH₃—C(═O)—CH₂—C(═O)—CH₃” as a basic structure and which is obtained by removing one hydrogen ion from this basic structure and coordinating the resulting basic structure to M with an oxygen atom as a coordinating atom. The expression “having an acetylacetone structure as a basic structure” is meant to include, besides the acetylacetone structure, structures in which a hydrogen atom in the acetylacetone structure is substituted with a substituent. Examples of the form in which M is Al and OR² has an acetonato structure include compounds A-2 and A-3 described below.

When M is Al, at least one of OR² in general formula (1) or (2) above preferably has an acetato structure. This acetato structure means a structure obtained by removing one hydrogen ion from an acetate or a compound having a structure of an acetate with a substituent (including an alkyl group) and coordinating the resulting structure to M. The coordinating atom that coordinates to M is usually an oxygen atom. This acetato structure is preferably a structure which has an alkyl acetoacetate structure “CH₃—C(═O)—CH₂—C(═O)—O—Rₐₗₖ (where Rₐₗₖ represents an alkyl group) as a basic structure and which is obtained by removing one hydrogen ion from this basic structure and coordinating the resulting basic structures to M with an oxygen atom as a coordinating atom. The expression “having an alkyl acetoacetate structure as a basic structure” is meant to include, besides the alkyl acetoacetate structure, structures in which a hydrogen atom in the alkyl acetoacetate structure is substituted with a substituent. Examples of the form in which M is Al and OR² has an acetato structure include compounds A-3, A-4, and A-5 described below.

Al is usually trivalent.

When M is Zr, at least one of OR² in general formula (1) or (2) above preferably has an acetonato structure. This acetonato structure has the same definition as in the acetonato structure described in the form in which M is Al. Examples of the form in which M is Zr and OR² has an acetonato structure include compounds Z-3 and Z-6 described below.

When M is Zr, at least one of OR² in general formula (1) or (2) above preferably has an acetato structure. This acetato structure has the same definition as in the acetato structure described in the form in which M is Al. An example of the form in which M is Zr and OR² has an acetato structure is compound Z-7 described below.

When M is Zr, at least one of OR² in general formula (1) or (2) above preferably has a lactato structure. This lactato structure means a structure which has a lactic acid ion (lactate) as a basic structure and which is obtained by removing one hydrogen ion from this basic structure and coordinating the resulting basic structure to M. The expression “having a lactic acid ion as a basic structure” is meant to include, besides the lactic acid ion, structures in which a hydrogen atom in the lactic acid ion is substituted with a substituent. The coordinating atom that coordinates to M is usually an oxygen atom. An example of the form in which M is Zr and OR² has a lactato structure is compound Z-4 described below.

When M is Zr, it is also preferable that at least one of R² in general formula (1) or (2) be an acyl group. An example of the form in which M is Zr and R² is an acyl group is compound Z-5 described below.

Zr is usually tetravalent.

Each of the groups that may be employed as R¹ or R² may have, as a substituent, an anionic group having a counter cation (substituent in the form of a salt). The term “anionic group” means a group capable of forming an anion. An example of the anionic group having a counter cation is a group of a carboxylic acid ion having an ammonium ion as a counter cation. In this case, the counter cation is present in the compound represented by general formula (1) or (2) above such that the electric charge of the whole compound is zero.

Specific examples of the compound represented by general formula (1) are described below. However, the present invention is not limited to these specific examples.

Examples in which M is Ti

Examples in which M is Ti include:
isopropyl triisostearoyl titanate,
isopropyl tridodecylbenzenesulfonyl titanate,
isopropyl trioctanoyl titanate,
isopropyl tri(dioctyl phosphite) titanate,
isopropyl tris(dioctylpyrophosphate) titanate,
isopropyl tri(dioctylsulfate) titanate,
isopropyl tricumylphenyl titanate,
isopropyl tri(N-aminoethyl-aminoethyl) titanate,
isopropyl dimethacrylisostearoyl titanate,
isopropyl isostearoyldiacryl titanate,
isobutyl trimethyl titanate,
diisostearoyl ethylene titanate,
diisopropyl bis(dioctylpyrophosphate) titanate,
dioctyl bis(ditridecylphosphite) titanate,
dicumyl phenyloxyacetate titanate,
bis(dioctylpyrophosphate)oxyacetate titanate,
bis(dioctylpyrophosphate)ethylene titanate,
tetraisopropyl titanate.
tetra-n-butyl titanate,
tetraoctyl titanate,
tetrastearyl titanate.
tetraisopropyl bis(dioctylphosphite) titanate.
tetraoctyl bis(di-tridecylphosphite) titanate,
tetra(2,2-diallyloxymethyl-1-butyl)bis(di-tridecyl)phosphite titanate,
butyl titanate dimer,
titanium tetraacetyl acetonate,
titanium ethylacetoacetate,
titanium octyleneglycolate, and
titanium di-2-ethylhexoxy bis(2-ethyl-3-hydroxyhexoxide).
   Examples in which M is Al

Examples in which M is Al include:
aluminum triethylate,
aluminum triisopropylate,
aluminum tri-sec-butylate,
aluminum tris(ethylacetoacetate),
ethylacetoacetate aluminum diisopropylate,
aluminum monoacetylacetonate bis(ethylacetoacetate),
aluminum tris(acetylacetonate),
diisopropoxy aluminum-9-octadecenylacetoacetate,
aluminum diisopropoxymonoethylacetoacetate,
mono-sec-butoxyaluminum diisopropylate,
ethylacetoacetate aluminum diisopropylate,
diethylacetoacetate aluminum isopropylate,
aluminum bisethylacetoacetate monoacetylacetonate,
aluminum trisethylacetoacetate, and
octadecylacetoacetate aluminum diisopropylate.
   Examples in which M is Zr

Examples in which M is Zr include:
tetra-n-propoxy zirconium (another name: zirconium tetra-n-propoxide),
tetra-n-butoxy zirconium (another name: zirconium teira-n-butoxide),
zirconium tetraacetylacetonate,
zirconium tributoxymonoacetylacetonate,
zirconium dibutoxybis(acetvlacetonate),
zirconium dibutoxybis(ethylacetoacetate),
zirconium tributoxyethylacetoacetate,
zirconium monobutoxyacetylacetonate bis(ethylacetoacetate),
zirconium tributoxy monostearate (another name: zirconium stearate tri-n-butoxide),
zirconium stearate,
zirconium lactate ammonium salt, and
zirconium monoacetylacetonate.

An example in which M is B is triethyl borate, an example in which M is Ba is barium acetylacetonate hydrate, an example in which M is Bi is bismuth tri-tert-amyloxide, an example in which M is Ca is calcium tert-butoxide, an example in which M is Ga is gallium triisopropoxide, an example in which M is Ge is germanium tetraethoxide, an example in which M is Hf is hafnium tetra-n-butoxide, an example in which M is In is indium triisopropoxide, an example in which M is La is lanthanum triisopropoxide, an example in which M is Mg is magnesium bis(2-methyl-2-propanolate), an example in which M is Nb is niobium penta-n-butoxide, an example in which M is P is trimethyl phosphate, an example in which M is Sr is strontium isopropoxide, an example in which M is Sn is tin-n-butoxide, an example in which M is Ta is tantalum penta-n-butoxide, an example in which M is V is vanadium tri-n-butoxideoxide, and an example in which M is Y is yttrium n-butoxide.

In the present invention, the expression “a primer layer includes at least one compound represented by general formula (1) or (2)” is meant to include a form in which the compound represented by general formula (1) or (2) is included in a state of having reacted with the flexible tube base and a form in which the compound represented by general formula (1) or (2) is included in a state of having reacted with the resin cover layer. Specifically, at least a portion of the compound represented by general formula (1) or (2) is hydrolyzed and a hydroxy group is thereby exposed, and the compound represented by general formula (1) or (2) can be present in a state where the exposed hydroxy group reacts with the metal constituting the flexible tube base or reacts with a group on the surface of the resin cover layer.

### Resin Cover Layer

The flexible tube according to the present invention has a resin cover layer on an outer periphery of a flexible tube base having a primer layer thereon.

In the embodiment in FIG. 2, an outer surface of a resin cover layer 15 is coated with a topcoat layer 16 that contains fluorine or the like and that contributes to, for example, chemical resistance. In FIG. 2, a spiral tube 11 is illustrated as a single layer. Alternatively, the spiral tube 11 may be formed by concentrically stacking two or more layers. Note that the resin cover layer 15 and the topcoat layer 16 in the figure are shown to be thicker than the actual thicknesses with respect to the diameter of a flexible tube base 14 for the sake of clearly illustrating the layer structure.

In the present invention, the resin cover layer covers an outer peripheral surface of the flexible tube base having the above-described primer layer thereon. The resin cover layer 15 in the embodiment in FIG. 2 has a two-layer structure in which an inner layer 17 that covers the entire peripheral surface around the axis of the flexible tube base 14 and an outer layer 18 that covers the entire peripheral surface around the axis of the inner layer 17 are stacked. Typically, a soft resin is used as the material of the inner layer 17, and a hard resin is used as the material of the outer layer 18. However, the present invention is not limited to these embodiments.

In the present invention, when the resin cover layer has a multilayer structure having two or more layers, at least the innermost layer (layer that is in contact with the primer layer) includes at least one compound selected from the group consisting of polyamides, polyesters, polyurethanes, and polyolefins, as described below. In the present invention, when the resin cover layer is formed of a single layer, this single-layer resin cover layer includes at least one compound selected from the group consisting of polyamides, polyesters, polyurethanes, and polyolefins. That is, the resin cover layer in the present invention includes at least one compound selected from the group consisting of polyamides, polyesters, polyurethanes, and polyolefins at least on the side of the resin cover layer in contact with the primer layer.

### Polyamide

Typical polyamides that can be used as a resin cover layer of a flexible tube for an endoscope can be widely employed as the polyamides. Examples thereof include crystalline polyamides, amorphous polyamides, and polyamide elastomers.

Examples of the crystalline polyamides include, but are not particularly limited to, aliphatic polyamides and aromatic polyamides.

Examples of the aliphatic polyamides include poly-s-caproamide (polyamide 6), polytetramethylene adipamide (polyamide 46), polyhexamethylene adipamide (polyamide 66), polycaproamide/polyhexamethylene adipamide copolymers (polyamide 6/66), polyundecamide (polyamide 11), polycaproamide/polyundecamide copolymers (polyamide 6/11), polydodecamide (polyamide 12), polycaporamide/polydodecamide copolymers (polyamide 6/12), polyhexamethylene sebacamide (polyamide 610), polydecamethylene sebacamide (polyamide 1010), polyhexamethylene dodecamide (polyamide 612), polydecamethylene dodecamide (polyamide 1012), polyundecamethylene adipamide (polyamide 116), and mixtures and copolymers thereof.

Examples of the aromatic polyamides include polyhexamethylene isophthalamide (polyamide 61), polyhexamethylene terephthalamide (polyamide 6T), polyhexamethylene terephthalamide/polyhexamethylene isophthalamide copolymers (polyamide 6T/6I), polycaproamide/polyhexamethylene terephthalamide copolymers (polyamide 6/6T), polycaproanide/polyhexamethylene isophthalamide copolymers (polyamide 6/6I), polyhexamethylene adipamide/polyhexamethylene terephthalamide copolymers (polyamide 66/6T), polyhexamethylene adipamide/polyhexamethylene isophthalamide copolymers (polyamide 66/6I), polytrimethylhexamethylene terephthalamide (polyamide TMDT), polybis(4-aminocyclohexyl)methane dodecamide (polyamide PACM12), polybis(3-methyl-4-aminocyclohexyl)methane dodecamide (nylon dimethyl PACM12), poly-m-xylylene adipamide (polyamide MXD6), polydecamethylene terephthalamide (polyamide 10T), polyundecamethylene terephthalanude (polyamide 11T), and mixtures and copolymers thereof.

Examples of the amorphous polyamides include polycondensates of isophthalic acid/terephthalic acid/1,6-hexanediamine/bis(3-methyl-4-aminocyclohexyl)methane, polycondensates of terephthalic acid/2,2,4-trimethyl-1,6-hexanediamine/2,4,4-trimethyl-1,6-hexanediamine, polycondensates of isophthalic acid/bis(3-methyl-4-aminocyclohexyl)methane/ω-laurolactam, polycondensates of isophthalic acid/terephthalic acid/1,6-hexanedianune, polycondensates of isophthalic acid/2,2,4-trimethyl-1,6-hexanediamine/2,4,4-trimethyl-1,6-hexanediamine, polycondensates of isophthalic acid/terephthalic acid/2,2,4-trimethyl-1,6-hexanediamine/2,4,4-trimethyl-1,6-hexanediamine, polycondensates of isophthalic acid/bis(3-methyl-4-aminocyclohexyl)methane/w-laurolactam, and polycondensates of isophthalic acid/terephthalic acid/other diamine components.

Examples of the polyamide elastomers include elastomers containing polyamides as hard segments, the elastomers being called amide-based thermoplastic elastomers. Examples thereof include multiblock copolymers having hard segments composed of polyamides and soft segments composed of polyethers or polyesters, and multiblock copolymers having hard segments composed of polyamides and soft segments having bonding forms of both an ether bond and an ester bond. Examples of the hard segments include polyamides 6, 66, 610, 11, and 12. Examples of the polyethers for the soft segments include polyethylene glycol, poly(oxytetramethylene) glycol, and poly(oxypropylene) glycol. Examples of the polyesters include poly(ethylene adipate) glycol and poly(butylene-1,4-adipate) glycol.

Examples of commercially available polyamides that can be used in the present invention include polyamide 11 (trade name “Rilsan BMN O” manufactured by Arkema Inc.), polyamide 12 (trade name “DAIAMID L1940” manufactured by Daicel-Evonik Ltd.), polyamide 1010 (trade name “VESTAMID Terra DS16” manufactured by Daicel-Evonik Ltd.), polyamide 1012 (trade name “VESTAMID Terra DD16” manufactured by Evonik), an amorphous polyamide (trade name “TROGAMID CX7323” manufactured by Daicel-Evonik Ltd.), and polyamide elastomers (trade name “PEBAX 7233” and “PEBAX Rnew 80R53” manufactured by Arkema Inc.).

These polyamides may be used alone or in combination of two or more thereof.

### Polyester

Typical polyesters that can be used as a resin cover layer of a flexible tube for an endoscope can be widely employed as the polyesters. Examples thereof include thermoplastic polyesters and polyester elastomers.

Examples of the thermoplastic polyesters include polyester resins formed from a dicarboxylic acid component and a diol component and polyester resins formed from a hydroxycarboxylic acid component.

Examples of the dicarboxylic acid component include terephthalic acid, isophthalic acid, phthalic acid, 2,6-naphthalenedicarboxylic acid, 4,4′-biphenvldicarboxylic acid, 5-sodiosulfoisophthalic acid, oxalic acid, succinic acid, adipic acid, sebacic acid, azelaic acid, dodecanedioic acid, dimer acids, maleic anhydride, maleic acid, fumaric acid, itaconic acid, citraconic acid, mesaconic acid, and cyclohexanedicarboxylic acid.

Examples of the diol component include ethylene glycol, diethylene glycol, 1,3-propanediol, 1,4-butanediol, neopentyl glycol, 1,6-hexanediol, cyclohexanedimethanol, triethylene glycol, polyethylene glycol, polypropylene glycol, polytetramethylene glycol, and ethylene oxide adducts of bisphenol A and bisphenol S.

Examples of the hydroxycarboxylic acid component include ε-caprolactone, lactic acid, and 4-hydroxybenzoic acid.

The thermoplastic polyester resins may be homopolymers formed from the dicarboxylic acid component and the diol component or homopolymers formed from the hydroxycarboxylic acid component, or copolymers formed from the above components. The thermoplastic polyester resins may further contain a small amount of a trifunctional or higher compound component such as trimellitic acid, trimesic acid, pyromellitic acid, trimethylolpropane, glycerol, or pentaerythritol.

Examples of the polyester elastomers include elastomers containing polyesters as hard segments, the elastomers being called ester-based thermoplastic elastomers. Examples thereof include multiblock copolymers having hard segments composed of crystalline polyesters and soft segments composed of polyethers or polyesters, and multiblock copolymers having hard segments composed of crystalline polyesters and soft segments having bonding forms of both an ether bond and an ester bond.

Examples of the hard segments include polybutylene terephthalate and polyethylene terephthalate.

Examples of the soft segments include polyalkylene glycols such as polytetramethylene glycol and polypropylene glycol, bisphenol A-ethylene oxide adducts, bisphenol A-propylene oxide adducts, and polyesters such as polycaprolactone.

For example, block copolymers composed of high-melting-point polyester segments (hard segments) and low-melting-point polymer segments (soft segments) having a molecular weight of 400 to 6,000 can be used as the polyester elastomers, as described in, for example, JP1999-92636A (JP-H11-92636A).

Examples of commercially available polyesters used in the present invention include polyester elastomers (trade name “PELPRENE P-70B” and “PELPRENE S-3001” manufactured by Toyobo Co., Ltd.) and (trade name “PRIMALLOY B1942” manufactured by Mitsubishi Chemical Corporation) and polybutylene terephthalate (trade name “NOVADURAN 55055” manufactured by Mitsubishi Engineering-Plastics Corporation).

These polyesters may be used alone or in combination of two or more thereof.

### Polyurethane

Typical polyurethanes that can be used as a resin cover layer of a flexible tube for an endoscope can be widely employed as the polyurethanes. For example, carbonate-based, ether-based, or ester-based poly urethanes, or mixed polyurethanes of these can be used. Polyurethane elastomers are also preferred. The polyurethane elastomers may be block polymers including hard segments composed of polyurethanes and soft segments having an ether, ester, or carbonate bond or a mixed form of these bonds, the block polymers being called urethane-based thermoplastic elastomers. Such polyurethane elastomers can be appropriately prepared depending on the purpose. Examples thereof include block polymers including hard segments composed of low-molecular-weight glycol components and diisocyanate components and soft segments composed of high-molecular-weight (long-chain) diol components and diisocyanate components.

Examples of the high-molecular-weight (long-chain) diol components include polyether diols, polyester diols, and lactone-based polyester diols. Examples thereof include polypropylene glycol, polytetramethylene oxide, poly(1,4-butylene adipate), poly(ethylene adipate-co-1,4-butylene adipate), polycaprolactone-based diol, poly(1,6-hexylene carbonate), and poly(1,6-hexylene adipate-co-neopentylene adipate). The high-molecular-weight (long-chain) diols preferably have a number-average molecular weight of 500 to 10.000.

As the low-molecular-weight glycol components, short-chain diols such as ethylene glycol, propylene glycol, 1,4-butanediol, and bisphenol A can be used. The short-chain diols preferably have a number-average molecular weight of 48 to 500.

Examples of the diisocyanate components include diphenylmethane diisocyanate, hexamethylene diisocyanate, tolidine diisocyanate, 1,5-naphthalene diisocyanate, isophorone diisocyanate, and xylylene diisocyanate.

For the polyurethane elastomers according to the above embodiment, disclosure of, for example, JP2005-015643A can be referred to.

Examples of commercially available polyurethanes that can be used in the present invention include PANDEX T-2185 and T-2983N (which are manufactured by DIC Corporation), Miractran (manufactured by Nippon Miractran Co., Ltd.), Elastollan (manufactured by BASF Japan Ltd.), RESAMINE (manufactured by Dainichiseika Color & Chemicals Mfg. Co., Ltd.). Pellethane (manufactured by Dow Chemical Japan Ltd.), Iron Rubber (manufactured by NOK Corporation), and Mobilon (manufactured by Nisshinbo Chemical Inc.). Examples thereof further include Isoplast (manufactured by Lubrizol Corporation). Tecoflex (manufactured by Lubrizol Corporation), Superflex 830, 460, 870, 420, and 420NS (polyurethanes manufactured by DKS Co., Ltd.), Hydran AP-40F, WLS-202, and HW-140SF (polyurethanes manufactured by DIC Corporation), Olester UD500 and UD350 (polyurethanes manufactured by Mitsui Chemicals. Inc.), and Takelac W-615. W-6010, W-6020, W-6061, W-405, W-5030, W-5661, W-512A-6, W-635, and WPB-6601 (manufactured by Mitsui Chemicals, Inc.).

These polyurethanes may be used alone or in combination of two or more thereof.

### Polyolefin

Typical polyolefins that can be used as a resin cover layer of a flexible tube for an endoscope can be widely employed as the polyolefins. Examples thereof include polyolefins and olefin-based elastomers.

Examples of the polyolefins include homopolymers and copolymers of α-olefins having 2 to 20 carbon atoms, such as ethylene, propylene, 1-butene, 1-hexene, and 4-methyl-pentene. Examples thereof further include copolymers of α-olefins and nonconjugated dienes having 2 to 20 carbon atoms, such as dicyclopentadiene, 1,4-hexadiene, cyclooctadiene, methylene norbornene, ethylidene norbomene, butadiene, and isoprene. Examples thereof further include ethylene-α-olefin copolymer rubbers, ethylene-α-olefin-nonconjugated diene copolymer rubbers, propylene-α-olefin copolymer rubbers, and butene-α-olefin copolymer rubbers. It is also possible to use, for example, ethylene-(meth)acrylic acid copolymers, ethylene-(meth)acrylic acid ester-(meth)acrylic acid copolymers, ethylene-vinyl acetate copolymers, ethylene-vinyl acetate-(meth)acrylic acid copolymers, ethylene-propylene-(meth)acrylic acid copolymers, ethylene-propylene-(meth)acrylic acid ester-(meth)acrylic acid copolymers, ethylene-maleic anhydride copolymers, ethylene-(meth)acrylic acid ester-maleic anhydride copolymers, ethylene-butene-maleic anhydride and/or (meth)acrylic acid copolymers, propylene-butene-maleic anhydride and/or (meth)acrylic acid copolymers, and ethylene-vinyl chloride copolymers.

Examples of polyolefins in the olefin-based elastomers include ethylene-propylene copolymers, ethylene-1-butene copolymers, ethylene-α-olefin copolymers, propylene-1-butene copolymers, propylene-α-olefin copolymers, 1-butene-α-olefin copolymers, propylene-1-butene-ethylene copolymers, propylene-α-olefin-ethylene copolymers, propylene-α-olefin-1-butene copolymers, 1-butene-α-olefin-ethylene copolymers, and polypropylene.

Examples of rubber components in the olefin-based elastomers include propylene rubber (PP), ethylene-propylene rubber (EPM), ethylene-propylene-diene rubber (EPDM), polvisoprene, polybutadiene, polychloroprene, and isobutylene-isoprene copolymers.

The olefin-based elastomers may contain one of the polyolefins alone or two or more of the polyolefins in combination and may contain one of the rubber components alone or two or more of the rubber components in combination.

Examples of commercially available polyolefin resins used in the present invention include an olefin-based elastomer (trade name “SARLINK 3145D” manufactured by Toyobo Co., Ltd.).

These polyolefins may be used alone or in combination of two or more thereof.

The total amount of compounds selected from the group consisting of polyamides, polyesters, polyurethanes, and polyolefins contained in the resin cover layer in the case of a single-layer resin cover layer, and the total amount of compounds selected from the group consisting of polyamides, polyesters, polyurethanes, and polyolefins contained in the innermost layer in the case of a multilayer resin cover layer are each preferably 50% by mass or more, more preferably 70% by mass or more, still more preferably 80% by mass or more, and still more preferably 90% by mass or more. When the resin cover layer is formed of a single layer, the resin cover layer may be a layer composed of at least one selected from the group consisting of polyamide resins, polyester resins, polyurethane resins, and polyolefin resins. When the resin cover layer is formed of a plurality of layers, the innermost layer may be a layer composed of at least one selected from the group consisting of polyamide resins, polyester resins, polyurethane resins, and polyolefin resins.

When the resin cover layer in the case of a single-layer resin cover layer and the innermost layer in the case of a multilayer resin cover layer include a polymer other than polymers selected from the group consisting of polyamides, polyesters, polyurethanes, and polyolefins, the polymer is not particularly limited as long as the effects of the present invention are not impaired.

The resin cover layer may appropriately contain various common additives as long as the effects of the present invention are not impaired. Examples of the additives include a heat-resistant stabilizer, a mineral filler, an impact resistance-improving agent, a plasticizer, a lubricant, a metal soap, a light-fast auxiliary agent, and a colorant. The contents of the additives in the resin cover layer can also be appropriately adjusted. Such additives may be derived from resin materials used or can be added separately from resins.

When the resin cover layer is formed of a plurality of layers, a layer other than the innermost layer preferably includes at least one compound selected from the group consisting of polyamides, polyesters, polyurethanes, and polyolefins. A layer having desired physical properties can be formed by appropriately combining these polymers.

Each of the polymers that can be used as the resin cover layer according to the present invention preferably has a molecular weight of 10,000 to 1,000,000, more preferably has a molecular weight of 20,000 to 500.000, and particularly preferably has a molecular weight of 30,000 to 300,000.

In the present invention, the molecular weight of the polymer that forms the resin cover layer means a weight-average molecular weight unless otherwise noted. The weight-average molecular weight can be measured by gel permeation chromatography (GPC) as a molecular weight in terms of polystyrene.

As illustrated in FIG. 2, the resin cover layer 15 in the present invention is preferably formed so as to have a substantially uniform thickness in the longitudinal direction (axial direction) of the flexible tube base 14. The resin cover layer 15 has a thickness of, for example, 0.2 mm to 1.0 mm. An outer diameter D of the flexible tube 3a is appropriately determined according to the purpose. The outer diameter D is, for example, 11 to 14 mm. In FIG. 2, the inner layer 17 and the outer layer 18 are formed such that a proportion of a thickness of the inner layer 17 to a total thickness of the resin cover layer 15 and a proportion of a thickness of the outer layer 18 to the total thickness of the resin cover layer 15 change in the axial direction of the flexible tube base 14. Specifically, on one end 14a side (distal end side) of the flexible tube base 14 to be attached to the angle portion 3b, the thickness of the inner layer 17 is larger than the thickness of the outer layer 18 with respect to the total thickness of the resin cover laver 15. The thickness of the inner layer 17 gradually decreases from the one end 14a toward the other end 14b side (proximal end side) to be attached to the main body operating section 5. On the other end 14b side, the thickness of the outer layer 18 is larger than the thickness of the inner layer 17.

In FIG. 2, the proportion of the thickness of the inner layer 17 is the maximum at the one end 14a, and the proportion of the thickness of the outer layer 18 is the maximum at the other end 14b. A ratio of the thickness of the inner layer 17 to the thickness of the outer layer 18 (thickness of inner layer 17:thickness of outer layer 18) can be, for example, 9:1 at the one end 14a, and, for example, 1:9 at the other end 14b. The thicknesses of the two layers are changed such that the ratio of the thickness of the inner layer 17 to the thickness of the outer layer 18 is reversed from the one end 14a to the other end 14b. With this configuration, the flexible tube 3a has a difference in hardness between the one end 14a side and the other end 14b side, and flexibility can be changed in the axial direction such that the one end 14a side is soft and the other end 14b side is hard. The inner layer and the outer layer are preferably formed such that the thickness ratio at the one end is 95:5 to 60:40 (inner layer:outer layer) and the thickness ratio at the other end is 5:95 to 40:60 (inner layer:outer layer).

When the ratio of the thickness of the inner layer 17 to the thickness of the outer layer 18 is within the range of 95:5 to 5:95, the amount of extrusion of a resin that forms a layer having a smaller thickness can also be accurately controlled.

A difference in 100% modulus, which is an indicator indicating a hardness after molding, between the soft resin used in the inner layer 17 and the hard resin used in the outer layer 18 is preferably 1 MPa or more and more preferably 3 MPa or more. A difference in melt viscosity at a molding temperature of 150° C. to 300° C., which is an indicator indicating the fluidity of a resin in a molten state, is preferably 2,500 Pa·s or less. With this configuration, the resin cover layer 15 formed of the inner layer 17 and the outer layer 18 reliably achieves both good molding accuracy and the necessary difference in hardness between the distal end side and the proximal end side.

In the present invention, from the viewpoint of further enhancing the adhesiveness between the flexible tube base and the resin cover layer, when compounds in which M in general formulae (1) and (2) is Ti are used for the primer layer, a polyamide or a polyester is preferably used for the resin cover layer that is in contact with this primer layer. In this case, the compounds in which M in general formulae (1) and (2) is Ti preferably include at least one atom selected from the group consisting of N, P, and S.

From the same viewpoint, when compounds in which M in general formulae (1) and (2) is Al are used for the primer layer, a polyolefin is preferably used for the resin cover layer that is in contact with this primer layer. In this case, the compounds in which M in general formulae (1) and (2) is Al preferably have at least one structure selected from the group consisting of an acetonato structure and an acetato structure.

From the same viewpoint, when compounds in which M in general formulae (1) and (2) is Zr are used for the primer layer, a polyurethane is preferably used for the resin cover layer that is in contact with this primer layer. In this case, the compounds in which M in general formulae (1) and (2) is Zr preferably have at least one structure selected from the group consisting of an acetonato structure, an acetato structure, and a lactato structure.

### Topcoat Layer

In the flexible tube according to the present invention, the topcoat layer 16 is disposed on an outer periphery of the resin cover layer 15 as needed. Examples of the material of the topcoat layer include, but are not particularly limited to, urethane coatings, acrylic coatings, fluorine coatings, silicone coatings, epoxy coatings, and polyester coatings.

Main purposes of use of the topcoat layer are to protect the surface of the flexible tube, to make the surface of the flexible tube glossy, to impart slidability, and to impart chemical resistance. Therefore, the topcoat layer is preferably formed of a material that has a high modulus of elasticity, that provides a smooth surface, and that has good chemical resistance.

### Method for Producing Flexible Tube

### Formation of Primer Layer

In the production of a flexible tube according to the present invention, first, a primer layer is formed on a flexible tube base. The primer layer can be formed by dissolving at least one of the compounds represented by general formula (1) or the compounds represented by general formula (2) in a solvent to prepare a coating liquid; forming a coating film on at least an outer periphery of the flexible tube base by, for example, applying or spraying the coating liquid onto the outer periphery of the flexible tube base or immersing the flexible tube base in the coating liquid; and subsequently drying the coating film by an ordinary method (for example, high-temperature drying at about 100° C.).

Examples of the solvent that can be used for the coating liquid include alcohol solvents such as methanol and ethanol; ketone solvents such as acetone and methyl ethyl ketone; ester solvents such as ethyl acetate; hydrocarbon solvents such as toluene; and liquid mixtures thereof. It is preferable to mix water or an acid catalyst, such as acetic acid, with the solvents in order to accelerate hydrolysis of the compound represented by general formula (1) or (2). The coating liquid may be adjusted to be acidic (for example, pH 1 to 4 at 25° C.) or alkaline (for example, pH 9 to 11 at 25° C.).

The content of the compound represented by general formula (1) or (2) in the coating liquid is not particularly limited, can be, for example, 0.01% by mass to 2% by mass, and is preferably 0.05% by mass or more and less than 1.5% by mass and more preferably 0.1% by mass or more and less than 1.0% by mass.

The coating liquid may include, for example, a surfactant and a catalyst besides the compound represented by general formula (1) or (2), the solvent, and a pH adjuster. The coating liquid is more preferably constituted by the compound represented by general formula (1) or (2) and the solvent.

In the present invention, a portion that is not covered with the primer layer may be present on the outer periphery of the flexible tube base as long as the effects of the present invention are not impaired (that is, a defect may be partially generated in the primer layer).

Prior to the formation of the primer layer, the flexible tube base is preferably cleaned by degreasing with an acid solution, an alkali solution, an aqueous solution of a surfactant, an organic solvent, or the like. After the cleaning, the flexible tube base is preferably further washed with water or hot water so that an acid, an alkali, a surfactant, and the like decrease from the surface of the base.

### Formation of Resin Cover Layer

The formation of the resin cover layer will be described using, as an example, a case where the resin cover layer has a two-layer structure.

A flexible tube including a resin cover layer that has a two-layer structure having an inner layer and an outer layer can be produced by, for example, melt-kneading and extruding, around the flexible tube base on which the primer layer has been formed, a first resin material (resin material including at least one selected from the group consisting of polyamide resins, polyester resins, polyurethane resins, and polyolefin resins) that forms the inner layer and a second resin material that forms the outer layer, thereby covering the flexible tube base.

In an embodiment in which a resin cover layer is formed of one layer or three or more layers, the resin cover layer can also be produced by appropriately changing the layer structure with reference to the method described below.

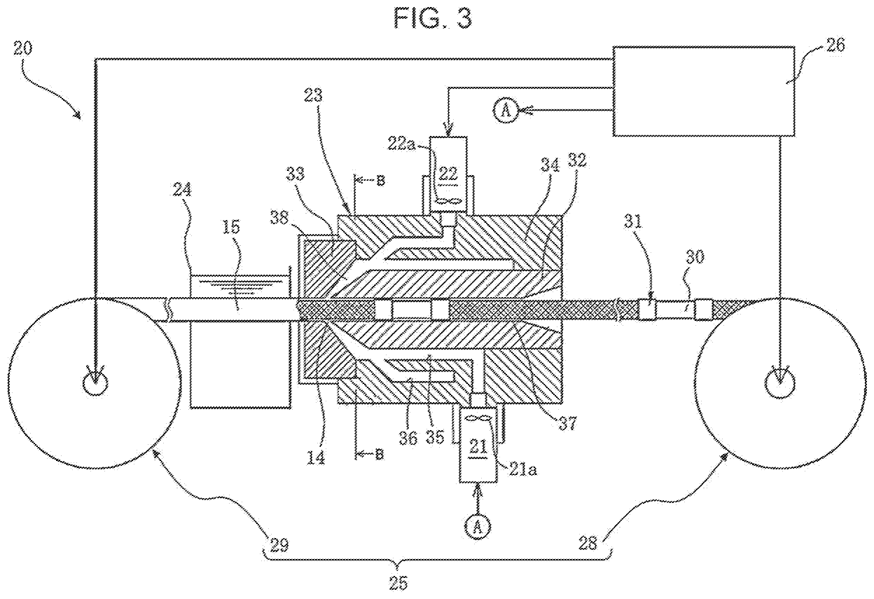

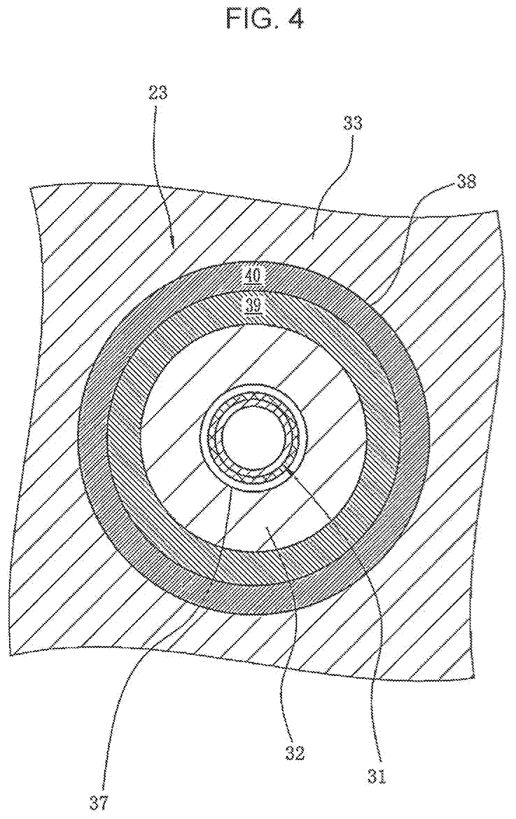

An example of a method for forming a resin cover layer of the flexible tube 3a (FIGS. 1 and 2) will be described with reference to FIGS. 3 and 4. In this embodiment, a continuous molding machine is used for molding a resin cover layer 15. It is preferable to use a continuous molding machine 20 that includes well-known extrusion units 21 and 22 including hoppers, screws 21a and 22a, etc.: a head unit 23 configured to mold a resin cover layer 15 so as to cover an outer peripheral surface of a flexible tube base 14; a cooling unit 24; a transport unit 25 (including a supply drum 28 and a take-up drum 29) configured to transport a connected flexible tube base 31 to the head unit 23; and a control unit 26 configured to control the above units. The head unit 23 preferably includes a nipple 32, a die 33, and a support 34 configured to support the nipple 32 and the die 33 in a fixed manner. For example, the apparatus disclosed in FIGS. 3 to 5 of JP2011-72391A can be used as an example of the apparatus having the above configuration.

The inside of the die 33 is preferably heated to a predetermined molding temperature. The molding temperature is preferably set in a range of 150° C. to 300° C. By controlling a temperature of a heating unit in the apparatus by heating, temperatures of a first resin material 39 and a second resin material 40 can be increased. In addition to this, as the rotation speeds of the screws 21a and 22a increase, the temperatures of the first resin material 39 and the second resin material 40 can be further increased to enhance the fluidity of the respective resin materials. In this case, the molding thicknesses of an inner layer 17 and an outer layer 18 can be respectively adjusted by changing the amounts of ejection of the first resin material 39 and the second resin material 40 in the molten state, while a transport speed of the connected flexible tube base 31 is made constant.

A process of molding the resin cover layer 15 on the connected flexible tube base 31 by the continuous molding machine 20 will be described. When the continuous molding machine 20 performs a molding process, the first resin material 39 and the second resin material 40 in the molten state are extruded from the extrusion units 21 and 22, respectively, to the head unit 23. Furthermore, the transport unit 25 operates so that the connected flexible tube base 31 is transported to the head unit 23. At this time, the extrusion units 21 and 22 are in a state of constantly extruding the first resin material 39 and the second resin material 40, respectively, to supply the resin materials 39 and 40 to the head unit 23, and the first resin material 39 and the second resin material 40 that are respectively extruded from the extrusion units 21 and 22 to gates 35 and 36 pass through edges and join to each other, and are supplied, in a stacked state, through a resin passage 38 to a molding passage 37. As a result, a two-layer molded resin cover layer 15 is formed in which an inner layer 17 composed of the first resin material 39 and an outer layer 18 composed of the second resin material 40 are stacked.

The connected flexible tube base 31 includes a plurality of flexible tube bases 14 (each having a primer layer on the outer periphery thereof) that are connected together. While the connected flexible tube base 31 is transferred in the molding passage 37, the resin cover layer 15 is continuously molded on the plurality of flexible tube bases 14. When the resin cover layer 15 is molded from one end 14a side (distal end side) of one flexible tube base to the other end 14b side (proximal end side) thereof, the thickness of the inner layer 17 is controlled to be large immediately after the extrusion units 21 and 22 start ejection of the resins. The proportion of the outer layer 18 is then gradually increased in an intermediate portion toward the other end 14b side. In this manner, the amounts of the resins ejected are preferably controlled such that the resin cover layer 15 has the thickness ratio that changes in a gradient manner.

Joint members 30 each function as a connecting portion of two flexible tube bases 14, and thus the joint members 30 are used for switching the amounts of resins ejected from the extrusion units 21 and 22 by the control unit 26. Specifically, the control unit 26 preferably switches the amounts of resins ejected from the extrusion units 21 and 22 such that the thickness ratio changes from a thickness ratio on the other end 14b side (proximal end side) of one flexible tube base 14 to a thickness ratio on one end 14a side (distal end side) of a next flexible tube base 14. When the resin cover layer 15 is molded from the one end 14a side of the next flexible tube base 14 to the other end 14b side thereof, the extrusion units 21 and 22 are preferably similarly controlled such that the thickness of the outer layer gradually increases from the one end side toward the other end side.

The connected flexible tube base 31 on which the resin cover layer 15 has been molded to the rearmost end is removed from the continuous molding machine 20. Subsequently, the joint members 30 are detached from the flexible tube bases 14 to separate the connected flexible tube base 31 into the individual flexible tube bases 14. Next, for each of the separated flexible tube bases 14, the resin cover layer 15 is coated with a topcoat layer 16 to complete flexible tubes 3a. The completed flexible tubes 3a are transferred to an assembly process of an electronic endoscope.

In the present invention, when the resin cover layer is formed of a plurality of layers, a functional layer may be disposed between layers that form the plurality of layers.

An electronic endoscope configured to observe an image of the condition of a subject captured by using an imaging device has been described with reference to the drawings by way of an example. However, the present invention is not limited thereto and is also applicable to an endoscope configured to examine the condition of a subject by employing an optical image guide.

The flexible tube according to the present invention is widely applicable to endoscopic medical devices. For example, the flexible tube according to the present invention is applicable to an endoscope equipped with a clip or wire at the distal end thereof or to an instrument equipped with a basket or a brush. Note that the term “endoscopic medical device” is meant to broadly include, besides the above-described medical devices that include an endoscope as a basic structure, medical devices and diagnosis and treatment devices that include an insertion section having flexibility and that are introduced into the body and used, such as remote-controlled medical devices.

An endoscopic medical device according to the present invention includes the flexible tube for an endoscope according to the present invention, the flexible tube being incorporated in an insertion section of the endoscopic medical device. That is, a method for producing an endoscopic medical device according to the present invention includes incorporating the flexible tube for an endoscope according to the present invention into an insertion section of an endoscopic medical device.

### EXAMPLES

Hereafter, the present invention will be described in more detail by way of Examples. However, it is to be understood that the present invention is not limited to these Examples.

### Preparation of Coating Liquid for Forming Primer Layer

A solution having a ratio water/ethanol of 5/75 on a mass basis was prepared. Each of the compounds shown in the tables below was separately dissolved in the solution so as to have a concentration of 8.9 g/kg. The resulting solutions were used as coating liquids for forming primer layers.

### Production of Flexible Tube for Endoscope

Flexible tubes having the structure illustrated in FIG. 2 were produced. The resin cover layers having a single-layer structure or a two-layer structure were formed by using the resin materials shown in tables below.

### Flexible Tube Base

Flexible tube bases were prepared. Each of the flexible tube bases had a form in which a spiral tube 11 was formed by using a metal strip 11a made of stainless steel, and the spiral tube 11 was covered with a tubular mesh 12 obtained by weaving stainless steel fibers. The flexible tube base has a length of 80 cm and a diameter of 12 mm. This stainless steel flexible tube base has a passivation layer on a surface thereof, the passivation layer being formed by annealing treatment (heating treatment) in the formation of the spiral tube and the tubular mesh.

### Formation of Primer Layer

The flexible tube bases were cleaned by immersing in a 7.5% aqueous solution of sodium hydroxide at 60° C. for one minute. Subsequently, the flexible tube bases were rinsed with distilled water and then dried in an oven at 100° C. for 10 minutes. The cleaned flexible tube bases were immersed in the above-prepared coating liquids for forming primer layers at room temperature for one minute and then dried in an oven at 160° C. for 10 minutes. Thus, flexible tube bases each having a primer layer on the outer periphery thereof (the surface to be covered with a resin) were prepared.

### Formation of Resin Cover Layer

The outer peripheries of the flexible tube bases having a primer layer thereon were covered with the resins shown in Table 1 below by extrusion (molding temperature: 200° C.) to produce flexible tubes for endoscopes, the flexible tubes having a resin cover layer. The resin cover layer had a thickness of 0.4 mm.

In the cases where the resin cover layer is formed of two layers (Examples 83 to 92), the two layers were simultaneously molded by two-layer extrusion molding to cover the flexible tube base to form a resin cover layer with a thickness of 0.4 mm. In these cases, a ratio of the inner layer to the outer layer, the ratio being represented by inner layer:outer layer, was 80:20 at the distal end and was 20:80 at the proximal end.

### Test Example 1

### Evaluation of Bending Durability

The above-produced flexible tube (length: 80 cm) for an endoscope was brought into contact, in a U shape, with a semicircular portion of a pulley with a diameter of 10 cm and reciprocated by alternately pulling one end and the other end of the U-shaped flexible tube for an endoscope. This reciprocating motion was performed such that a portion of the flexible tube for an endoscope, the portion having a length of 44. 3 cm and excluding portions each having a length of 17.85 cm from both ends of the flexible tube, successively formed the apex of the U shape while being in contact with the pulley. The number of reciprocating motions in which a crease, floating, tearing, or separation of a resin occurred was evaluated in accordance with the evaluation criteria described below.

### Evaluation Criteria for Bending Durability

A: 10,000 times or more

B: 1,000 times or more and less than 10,000 times

C: 100 times or more and less than 1,000 times

D: less than 100 times

The results are shown in the tables below.

### Test Example 2

### Evaluation of Ethylene Oxide Gas (EOG) Sterilization Resistance

Both ends of the above-produced flexible tube for an endoscope were capped with Teflon (registered trademark) plugs, and the flexible tube was sterilized by using an ethylene oxide gas sterilization device (Model EQ-150, manufactured by Miura Co., Ltd.). The sterilization conditions are described below.

### Sterilization Conditions

Ethylene oxide gas:Carbon dioxide=20:80

55° C.

50% RH

Reduction in pressure 71 kPa

Application of pressure 69 kPa

Gas concentration 450 mg/L

Pretreatment 1 hour

Sterilization treatment 5 hours

Ventilation (at 55° C.) after sterilization 12 hours

For the resin cover layer of the flexible tube for an endoscope after the EOG sterilization, a cut having a length of 5 cm and a width of 1 cm and extending in the axial direction of the flexible tube was formed in a direction perpendicular to the resin cover layer such that the cut reached the flexible tube base. For one end of this cut, a cut was further formed in the width direction to form a holding portion for a peeing test. The length direction of the cut formed above is the same as the axial direction of the flexible tube, and the cut has a width of 1 cm on the outer peripheral surface of the resin cover layer. A 90° peel strength was measured between the flexible tube base and the resin cover layer by peeling off the resin cover layer along the cut with a width of 1 cm in the axial direction at a constant speed while the angle between the flexible tube base and the peeled resin cover layer was maintained at 90° (measurement value X). The peel strength was measured with a force gauge (unit: N/cm).

The 90° peel strength was measured by using each flexible tube for an endoscope, the flexible tube not being subjected to the EOG sterilization treatment (endoscope flexible tube that was not subjected to EOG sterilization treatment), in the same manner (measurement value Y). For all the flexible tubes, the 90° peel strength was measured under the same conditions.

A ratio (%, 100×X/Y) of the measurement value X to the measurement value Y was determined, and an evaluation was performed in accordance with the criteria described below.

### Evaluation Criteria for EOG Sterilization Resistance

A: 80% or more

B: 60% or more and less than 80%

C: 40% or more and less than 60%

D: less than 40%

The results are shown in the tables below.

TABLE 1-1

| | | Example | Example | Example | Example | Example | Example | Example | Example | Example | Example |
|---|---|---|---|---|---|---|---|---|---|---|---|
| | | 1 | 2 | 3 | 4 | 5 | 6 | 7 | 8 | 9 | 10 |
| Structure | Compound | T-1 | T-2 | T-3 | T-4 | T-5 | T-6 | T-7 | T-8 | T-9 | T-10 |
| | of primer | | | | | | | | | | |
| | layer | | | | | | | | | | |
| | Resin of | PA1 | PA1 | PA1 | PA1 | PA1 | PA1 | PA1 | PA1 | PA1 | PA1 |
| | resin cover | | | | | | | | | | |
| | layer | | | | | | | | | | |
| Evaluation | Bending | B | B | B | A | A | A | A | A | A | C |
| | durability | | | | | | | | | | |
| | EOG | B | C | B | A | A | A | A | A | A | B |
| | sterilization | | | | | | | | | | |
| | resistance | | | | | | | | | | |

TABLE 1-2

| | | Example | Example | Example | Example | Example | Example | Example | Example | Example | Example |
|---|---|---|---|---|---|---|---|---|---|---|---|
| | | 11 | 12 | 13 | 14 | 15 | 16 | 17 | 18 | 19 | 20 |
| Structure | Compound | A-1 | A-2 | A-3 | A-4 | A-5 | Z-1 | Z-2 | Z-3 | Z-4 | Z-5 |
| | of primer | | | | | | | | | | |
| | layer | | | | | | | | | | |
| | Resin of | PA1 | PA1 | PA1 | PA1 | PA1 | PA1 | PA1 | PA1 | PA1 | PA1 |
| | resin cover | | | | | | | | | | |
| | layer | | | | | | | | | | |
| Evaluation | Bending | C | A | A | A | B | C | C | A | B | A |
| | durability | | | | | | | | | | |
| | EOG | C | B | B | B | A | C | C | B | A | B |
| | sterilization | | | | | | | | | | |
| | resistance | | | | | | | | | | |

TABLE 1-3

| | | Example | Example | Example | Example | Example | Example | Example | Example | Example | Example |
|---|---|---|---|---|---|---|---|---|---|---|---|
| | | 21 | 22 | 23 | 24 | 25 | 26 | 27 | 28 | 29 | 30 |
| Structure | Compound | Z-6 | Z-7 | T-5 | T-5 | T-5 | T-5 | T-5 | T-5 | T-5 | T-5 |
| | of primer | | | | | | | | | | |
| | layer | | | | | | | | | | |
| | Resin of | PA1 | PA1 | PA2 | PA3 | PA4 | TPEE1 | TPEE2 | TPEE3 | TPEE4 | TPU1 |
| | resin cover | | | | | | | | | | |
| | layer | | | | | | | | | | |
| Evaluation | Bending | B | B | A | A | A | A | A | A | A | A |
| | durability | | | | | | | | | | |
| | EOG | A | A | A | A | A | A | A | A | A | B |
| | sterilization | | | | | | | | | | |
| | resistance | | | | | | | | | | |

TABLE 1-4

| | | Example | Example | Example | Example | Example | Example | Example | Example | Example | Example |
|---|---|---|---|---|---|---|---|---|---|---|---|
| | | 31 | 32 | 33 | 34 | 35 | 36 | 37 | 38 | 39 | 40 |
| Structure | Compound | T-5 | T-5 | T-5 | A-3 | A-3 | A-3 | A-3 | A-3 | A-3 | A-3 |
| | of primer | | | | | | | | | | |
| | layer | | | | | | | | | | |
| | Resin of | TPU2 | TPU3 | PO1 | PA2 | PA3 | PA4 | TPEE1 | TPEE2 | TPEE3 | TPEE4 |
| | resin cover | | | | | | | | | | |
| | layer | | | | | | | | | | |
| Evaluation | Bending | A | A | B | B | A | B | A | A | A | A |
| | durability | | | | | | | | | | |
| | EOG | B | B | A | A | B | A | B | B | B | B |
| | sterilization | | | | | | | | | | |
| | resistance | | | | | | | | | | |

TABLE 1-5

| | | Example | Example | Example | Example | Example | Example | Example | Example | Example | Example |
|---|---|---|---|---|---|---|---|---|---|---|---|
| | | 41 | 42 | 43 | 44 | 45 | 46 | 47 | 48 | 49 | 50 |
| Structure | Compound | A-3 | A-3 | A-3 | A-1 | A-2 | A-3 | A-4 | A-5 | Z-3 | Z-3 |
| | of primer | | | | | | | | | | |
| | layer | | | | | | | | | | |
| | Resin of | TPU1 | TPU2 | TPU3 | PO1 | PO1 | PO1 | PO1 | PO1 | PA2 | PA3 |
| | resin cover | | | | | | | | | | |
| | layer | | | | | | | | | | |
| Evaluation | Bending | A | A | A | C | A | A | A | A | B | B |
| | durability | | | | | | | | | | |
| | EOG | B | B | B | C | A | A | A | A | A | A |
| | sterilization | | | | | | | | | | |
| | resistance | | | | | | | | | | |

TABLE 1-6

| | | Example | Example | Example | Example | Example | Example | Example | Example | Example | Example |
|---|---|---|---|---|---|---|---|---|---|---|---|
| | | 51 | 52 | 53 | 54 | 55 | 56 | 57 | 58 | 59 | 60 |
| Structure | Compound | Z-3 | Z-3 | Z-3 | Z-3 | Z-3 | Z-1 | Z-2 | Z-3 | Z-4 | Z-5 |
| | of primer | | | | | | | | | | |
| | layer | | | | | | | | | | |
| | Resin of | PA4 | TPEE1 | TPEE2 | TPEE3 | TPEE4 | TPU1 | TPU1 | TPU1 | TPU1 | TPU1 |
| | resin cover | | | | | | | | | | |
| | layer | | | | | | | | | | |
| Evaluation | Bending | B | A | A | A | A | C | C | A | A | A |
| | durability | | | | | | | | | | |
| | EOG | A | B | B | B | B | B | B | A | A | A |
| | sterilization | | | | | | | | | | |
| | resistance | | | | | | | | | | |

TABLE 1-7

| | | Example | Example | Example | Example | Example | Example | Example | Example | Example | Example |
|---|---|---|---|---|---|---|---|---|---|---|---|
| | | 61 | 62 | 63 | 64 | 65 | 66 | 67 | 68 | 69 | 70 |
| Structure | Compound | Z-6 | Z-7 | Z-3 | Z-3 | Z-3 | B-1 | Ba-1 | Bi-1 | Ca-1 | Ga-1 |
| | of primer | | | | | | | | | | |
| | layer | | | | | | | | | | |
| | Resin of | TPU1 | TPU1 | TPU2 | TPU3 | PO1 | PA1 | PA1 | PA1 | PA1 | PA1 |
| | resin cover | | | | | | | | | | |
| | layer | | | | | | | | | | |
| Evaluation | Bending | A | A | A | A | A | B | B | C | C | C |
| | durability | | | | | | | | | | |
| | EOG | A | A | A | A | B | C | C | C | B | C |
| | sterilization | | | | | | | | | | |
| | resistance | | | | | | | | | | |

TABLE 1-8

| | | Example | Example | Example | Example | Example | Example | Example | Example | Example | Example |
|---|---|---|---|---|---|---|---|---|---|---|---|
| | | 71 | 72 | 73 | 74 | 75 | 76 | 77 | 78 | 79 | 80 |
| Structure | Compound | Ge-1 | Hf-1 | In-1 | La-1 | Mg-1 | Nb-1 | P-1 | Sr-1 | Sn-1 | Ta-1 |
| | of primer | | | | | | | | | | |
| | layer | | | | | | | | | | |
| | Resin of | PA1 | PA1 | PA1 | PA1 | PA1 | PA1 | PA1 | PA1 | PA1 | PA1 |
| | resin cover | | | | | | | | | | |
| | layer | | | | | | | | | | |
| Evaluation | Bending | C | C | C | C | B | C | B | C | C | C |
| | durability | | | | | | | | | | |
| | EOG | B | C | B | C | C | C | C | B | C | C |
| | sterilization | | | | | | | | | | |
| | resistance | | | | | | | | | | |

TABLE 1-9

| | | | | Compar- | Compar- | Compar- | Compar- | Compar- | Compar- | Compar- | Compar- |
|---|---|---|---|---|---|---|---|---|---|---|---|
| | | | | ative | ative | ative | ative | ative | ative | ative | ative |
| | | Example | Example | Example | Example | Example | Example | Example | Example | Example | Example |
| | | 81 | 82 | 1 | 2 | 3 | 4 | 5 | 6 | 7 | 8 |
| Structure | Compound | V-1 | Y-1 | | | | R-1 | R-2 | R-1 | R-1 | R-1 |
| | of primer | | | | | | | | | | |
| | layer | | | | | | | | | | |
| | Resin of | PA1 | PA1 | PA1 | TPEE1 | PO1 | PA1 | PA1 | TPEE1 | TPU1 | PO1 |
| | resin cover | | | | | | | | | | |
| | layer | | | | | | | | | | |
| Evaluation | Bending | B | B | D | D | D | C | C | C | C | D |
| | durability | | | | | | | | | | |
| | EOG | C | C | D | D | D | D | D | D | D | C |
| | sterilization | | | | | | | | | | |
| | resistance | | | | | | | | | | |

TABLE 1-10

| | | Example | Example | Example | Example | Example | Example | Example | Example | Example | Example |
|---|---|---|---|---|---|---|---|---|---|---|---|
| | | 83 | 84 | 85 | 86 | 87 | 88 | 89 | 90 | 91 | 92 |
| Structure | Compound | T-1 | T-2 | T-3 | T-4 | T-5 | A-1 | A-2 | A-3 | Z-3 | Z-4 |
| | of primer | | | | | | | | | | |
| | layer | | | | | | | | | | |
| | Resin of | TPU1 | TPU1 | TPU1 | TPU1 | TPU1 | TPU1 | TPU1 | TPU1 | TPU1 | TPU1 |
| | resin inner | | | | | | | | | | |
| | cover layer | | | | | | | | | | |
| | Resin of | PA1 | PA1 | PA1 | PA1 | PA1 | PA1 | PA1 | PA1 | PA1 | PA1 |
| | resin outer | | | | | | | | | | |
| | cover layer | | | | | | | | | | |
| Evaluation | Bending | A | A | A | A | A | A | A | A | A | A |
| | durability | | | | | | | | | | |
| | EOG | B | C | B | A | A | C | B | B | B | A |
| | sterilization | | | | | | | | | | |
| | resistance | | | | | | | | | | |

The abbreviations shown in the above tables are as follows.

### <T-1>

Tetra-n-butyl titanate (“ORGATIX TA-21” manufactured by Matsumoto Fine Chemical Co., Ltd.)

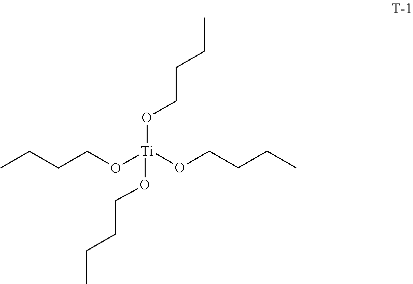

### <T-2>

n-Butyl titanate dimer (“ORGATIX TA-23” manufactured by Matsumoto Fine Chemical Co., Ltd.)

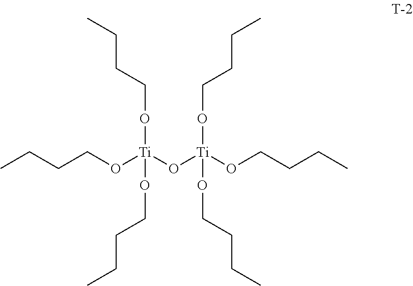

### <T-3>

Isopropyl triisostearoyl titanate (“PLENACT TTS” manufactured by Ajinomoto Fine-Techno Co., Inc.)

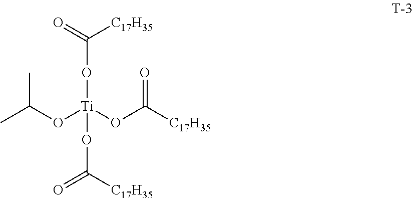

### <T-4>

Dioctylbis(ditridecyl)phosphate titanate (“PLENACT 46B” manufactured by Ajinomoto Fine-Techno Co., Inc.)

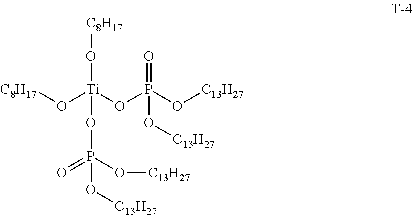

### <T-5>

Diisopropyl bis(dioctylpyrophosphate) titanate (“PLENACT 38S” manufactured by Ajinomoto Fine-Techno Co., Inc.)

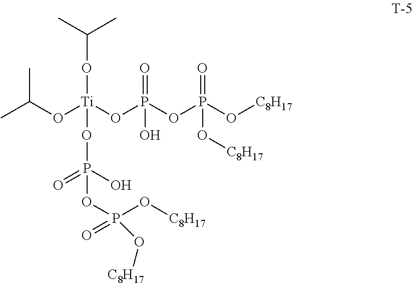

### <T-6>

Bis(dioctylpyrophosphate)oxyacetate titanate (“PLENACT 138S” manufactured by Ajinomoto Fine-Techno Co., Inc.)

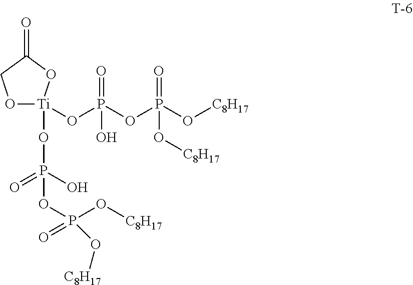

### <T-7>

Bis(dioctylpyrophosphate)ethylene titanate (“PLENACT 238S” manufactured by Ajinomoto Fine-Techno Co., Inc.)

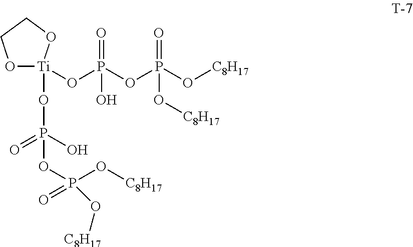

### <T-8>

Isopropyl tri(N-aminoethyl-aminoethyl) titanate (“PLENACT 44” manufactured by Ajinomoto Fine-Techno Co., Inc.)

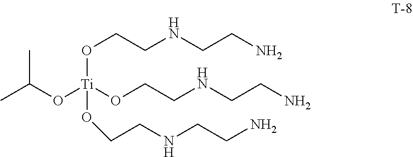

### <T-9>

Isopropyl tridodecylbenzenesulfonyl titanate (“PLENACT 9SA” manufactured by Ajinomoto Fine-Techno Co., Inc.)

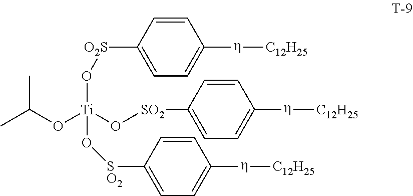

### <T-10>

Titanium di-2-ethylhexoxy bis(2-ethyl-3-hydroxyhexoxide) (“ORGATIX TC-201” manufactured by Matsumoto Fine Chemical Co., Ltd.)

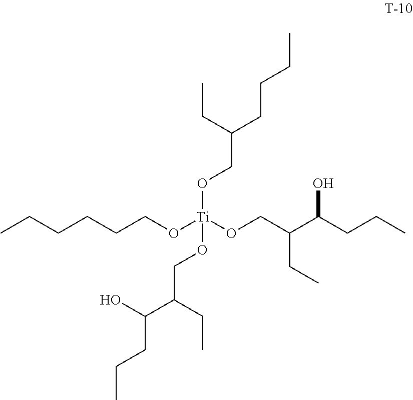

### <A-1>

Aluminum sec-butoxide (“ASBD” manufactured by Kawaken Fine Chemicals Co., Ltd.)

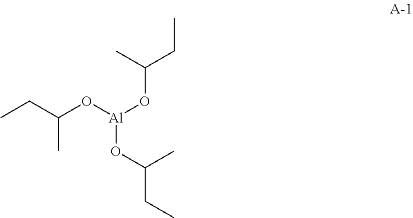

### <A-2>

Aluminum trisacetylacetonate (“ORGATIX AL-3100” manufactured by Matsumoto Fine Chemical Co., Ltd.)

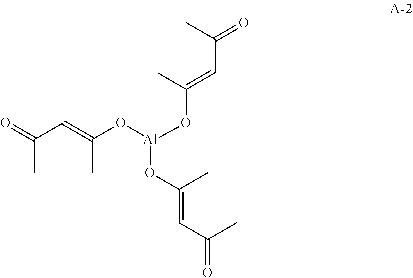

### <A-3>

Aluminum bisethylacetoacetate monoacetylacetonate (“ORGATIX AL-3200” manufactured by Matsumoto Fine Chemical Co., Ltd.)

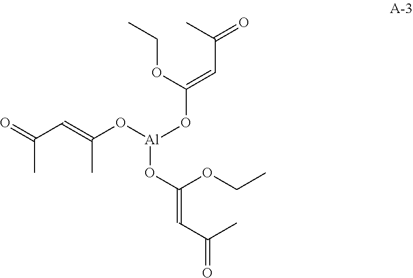

### <A-4>

Aluminum trisethylacetoacetate (“ORGATIX AL-3215” manufactured by Matsumoto Fine Chemical Co., Ltd.)

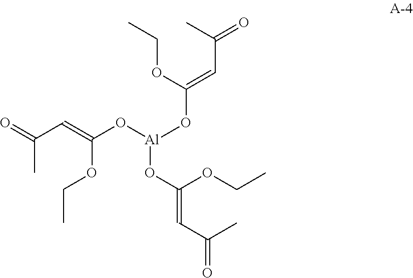

### <A-5>

Octadecylacetoacetate aluminum diisopropylate (“PLENACT AL-M” manufactured by Ajinomoto Fine-Techno Co., Inc.)

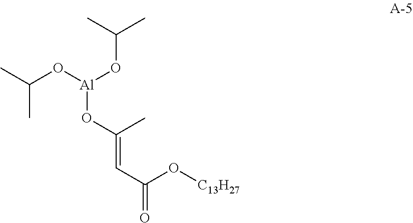

### <Z-1>

Zirconium tetra-n-propoxide (“ORGATIX ZA-45” manufactured by Matsumoto Fine Chemical Co., Ltd.)

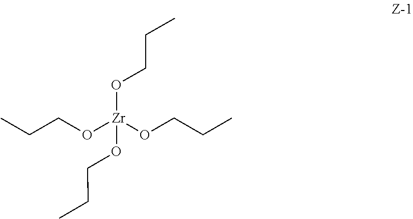

### <Z-2>

Zirconium tetra-n-butoxide (“ORGATIX ZA-65” manufactured by Matsumoto Fine Chemical Co., Ltd.)

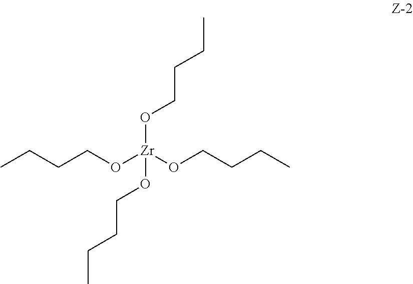

### <Z-3>

Zirconium tetraacetylacetonate (“ORGATIX ZC-150” manufactured by Matsumoto Fine Chemical Co., Ltd.)

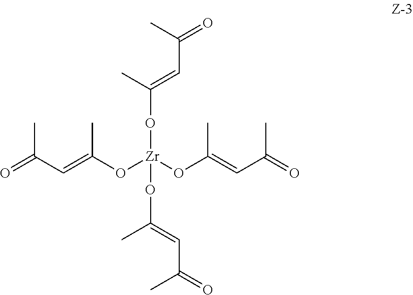

### <Z-4>

Zirconium lactate ammonium salt (“ORGATIX ZC-300” manufactured by Matsumoto Fine Chemical Co., Ltd.)

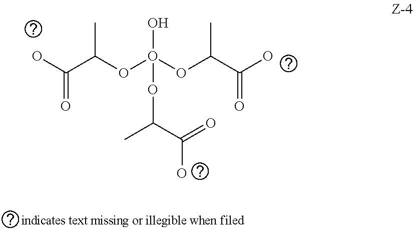

### <Z-5>

Zirconium stearate tri-n-butoxide (“ORGATIX ZC-320” manufactured by Matsumoto Fine Chemical Co., Ltd.)

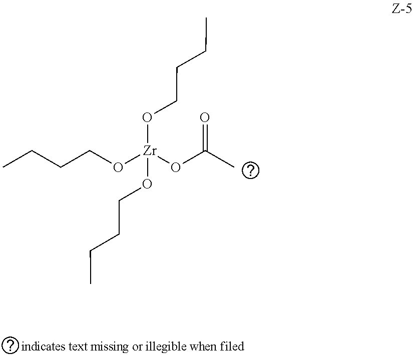

### <Z-6>

Zirconium tri-n-butoxy monoacetylacetonate (“ORGATIX ZC-540” manufactured by Matsumoto Fine Chemical Co., Ltd.)

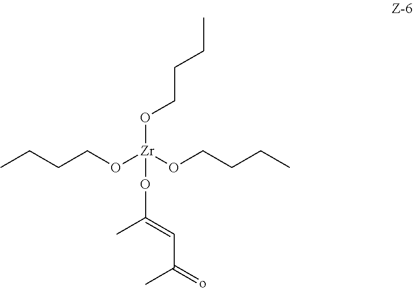

Zirconium di-n-butoxybis(ethylacetoacetate) (“ORGATIX ZC-580” manufactured by Matsumoto Fine Chemical Co., Ltd.)

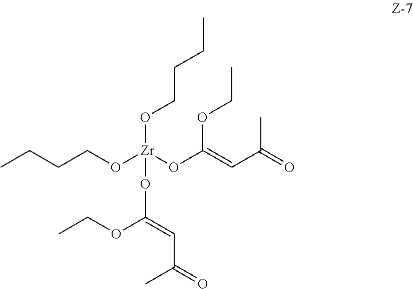

### <B-1>

Triethyl borate (manufactured by FUJIFILM Wako Pure Chemical Corporation)

### <Ba-1>

Barium acetylacetonate hydrate (manufactured by FUJIFILM Wako Pure Chemical Corporation)

### <Bi-1>

Bismuth tri-tert-amyloxide (manufactured by Kojundo Chemical Laboratory Co., Ltd.)

### <Ca-1>

Calcium tert-butoxide (manufactured by Tokyo Chemical Industry Co., Ltd.)

### <Ga-1>

Gallium triisopropoxide (manufactured by Kojundo Chemical Laboratory Co., Ltd.)

### <Ge-1>

Germanium tetraethoxide (manufactured by Hokko Chemical Industry Co., Ltd.)

### <Hf-1>

Hafnium tetra-n-butoxide (manufactured by Hokko Chemical Industry Co., Ltd.)

### <In-1>

Indium triisopropoxide (manufactured by Kojundo Chemical Laboratory Co., Ltd.)

### <La-1>

Lanthanum triisopropoxide (manufactured by Hokko Chemical Industry Co., Ltd.)

### <Mg-1>

Magnesium bis(2-methyl-2-propanolate) (manufactured by Capotchemical Co., Ltd.)

### <Nb-1>

Niobium penta-n-butoxide (manufactured by Hokko Chemical Industry Co., Ltd.)

### <P-1>

Trimethyl phosphate (manufactured by Kojundo Chemical Laboratory Co., Ltd.)

### <Sr-1>

Strontium isopropoxide (manufactured by Hokko Chemical Industry Co., Ltd.)

### <Sn-1>

Tin-n-butoxide (manufactured by Hokko Chemical Industry Co., Ltd.)

### <Ta-1>

Tantalum penta-n-butoxide (manufactured by Hokko Chemical Industry Co., Ltd.)

### <V-1>

Vanadium tri-n-butoxideoxide (manufactured by Kojundo Chemical Laboratory Co., Ltd.)

### <Y-1>

Yttrium n-butoxide (“HY-OB” manufactured by Hokko Chemical Industry Co., Ltd.)

### <R-1>

Vinyltnmethoxysilane

### <R-2>

Tetraethoxysilane

### Polyamide Resin

### <PA1>

“DAIAMID L1940” manufactured by Daicel-Evonik Ltd. (polyamide 12, melt volume rate (MVR)=8 cm³/10 min)

### <PA2>

“VESTAMID DS16” manufactured by Daicel-Evonik Ltd. (polyamide 1010, MVR=20 cm³/10 min)

### <PA3>

“Rilsan BMN O” manufactured by Arkema Inc. (polyamide 11. MVR=36 cm³/10 min)

### <PA4>

“PEBAX 7233” manufactured by Arkema Inc. (polyether block amide, MV R=4 cm³/10 min)

### Polyester Resin

### <TPEE1>

“PELPRENE P-70B” manufactured by Toyobo Co., Ltd. (MVR=20 cm³/10 min)

### <TPEE2>

“PELPRENE 5-3001” manufactured by Toyobo Co., Ltd. (MVR=16 cm³/10 min)

### <TPEE3>

“PRIMALLOY B1942” manufactured by Mitsubishi Chemical Corporation (MVR=59 cm³/10 min)

### <TPEE4>

“NOVADURAN 5505S” manufactured by Mitsubishi Engineering-Plastics Corporation (MVR=25 cm³/10 min)

### Polyurethane Resin

### <TPU1>

“Miractran E675” manufactured by Nippon Miractran Co., Ltd.

### <TPU2>

“PANDEX T-8185” manufactured by DIC Covestro Polymer Ltd. (MVR=18 cm³/10 min)

### <TPU3>

“PANDEX T-2190” manufactured by DIC Covestro Polymer Ltd. (MVR=12 cm³/10 min)

### Polyolefin Resin

### <PO1>

“SARLINK 3145D” manufactured by Toyobo Co., Ltd. (MVR=54 cm³/10 mm)

As shown in the tables above, it was found that in the cases where a resin cover layer was formed on the outer periphery of a flexible tube base without providing a primer layer, a crease, floating, tearing, or separation was easily caused when a bending load was repeatedly applied to the flexible tube and that adhesiveness between the flexible tube base and the resin cover layer was easily decreased by the EOG sterilization treatment (Comparative Examples 1 to 3).

In the cases where a compound that was not included in general formula (1) or (2) was used as a primer layer, adhesiveness between the flexible tube base and the resin cover layer tended to be impaired when a bending load was repeatedly applied to the flexible tube or when the flexible tube was subjected to the EOG sterilization treatment (Comparative Examples 4 to 8).

It was found that, in contrast, in the cases where a compound represented by general formula (1) or (2) was used as a primer layer, adhesiveness between the flexible tube base and the resin cover layer could be sufficiently maintained even when a bending load was repeatedly applied to the resulting flexible tube or when the flexible tube was subjected to the EOG sterilization treatment (Examples 1 to 92).

The present invention has been described together with embodiments thereof. However, we do not intend to limit our invention in any of the details of the description unless otherwise specified. We believe that the invention should be broadly construed without departing from the spirit and scope of the invention as defined by the appended claims.

### REFERENCE SIGNS LIST

2 electronic endoscope (endoscope)
3 insertion section
   3a flexible tube
   3b angle portion
   3c tip portion
5 main body operating section
6 universal cord
11 spiral tube
   11a metal strip
12 tubular mesh
13 cap
14 flexible tube base
   14a distal end side
   14b proximal end side
15 resin cover layer
16 topcoat layer
17 inner layer
18 outer layer
X angle portion 3b side (soft)
Y main body operating section 5 side (hard)
20 continuous molding machine (production apparatus)
21, 22 extrusion unit
   21a screw
   22a screw
23 head unit
24 cooling unit
25 transport unit
26 control unit
28 supply drum
29 take-up drum
30 joint member
31 connected flexible tube base
32 nipple
33 die
34 support
35, 36 gate
37 molding passage
38 resin passage
39 first resin material (soft resin)
40 second resin material (hard resin)

What is claimed is:

1. A flexible tube for an endoscope, the flexible tube comprising:
a flexible tube base containing metal as a constituent material;
a resin cover layer that covers an outer periphery of the flexible tube base; and
a primer layer that includes at least one compound represented by general formula (1) or (2) and that is disposed between the flexible tube base and the resin cover layer,
wherein the resin cover layer includes at least one compound selected from the group consisting of polyamides, polyesters, polyurethanes, and polyolefins at least on a side of the resin cover layer in contact with the primer layer:
R¹ₘ-M-(OR²)ₙ₋ₘ General formula (1):
O-[M-(OR²)ₙ₋₁]₂ General formula (2):
where M represents Al, B, Ba, Bi, Ca, Ga, Ge, Hf, In, La, Mg, Nb, P, Sr, Sn, Ta, Ti, V, Y, or Zr;
R¹ represents a hydrogen atom, an alkyl group, a cycloalkyl group, an acyl group, an aryl group, or an unsaturated aliphatic group;
R² represents a hydrogen atom, an alkyl group, a cycloalkyl group, an acyl group, an alkenyl group, an aryl group, a phosphonate group, or —SO₂R^{s} where R^{s} represents a substituent;
m is an integer of 0 to 3; n is a valence of M; and n>m is satisfied.

2. The flexible tube for an endoscope according to claim 1, wherein the M is Ti.

3. The flexible tube for an endoscope according to claim 2, wherein the compound represented by general formula (1) or (2) includes at least one atom selected from the group consisting of N, P, and S.

4. The flexible tube for an endoscope according to claim 1, wherein the M is Al.

5. The flexible tube for an endoscope according to claim 4, wherein at least one of OR² in general formulae (1) or (2) has an acetonato structure.

6. The flexible tube for an endoscope according to claim 4, wherein at least one of OR² in general formulae (1) or (2) has an acetato structure.

7. The flexible tube for an endoscope according to claim 1, wherein the M is Zr.

8. The flexible tube for an endoscope according to claim 7, wherein at least one of OR² in general formulae (1) or (2) has an acetonato structure.

9. The flexible tube for an endoscope according to claim 7, wherein at least one of OR² in general formulae (1) or (2) has an acetato structure.

10. The flexible tube for an endoscope according to claim 7, wherein at least one of OR² in general formulae (1) or (2) has a lactato structure.

11. The flexible tube for an endoscope according to claim 1, wherein the metal that constitutes the flexible tube base is stainless steel.

12. The flexible tube for an endoscope according to claim 1, wherein the metal that constitutes the flexible tube base has a passivation film on a surface thereof.

13. The flexible tube for an endoscope according to claim 1, wherein the resin cover layer has a single-layer structure or a multilayer structure and includes at least one compound selected from the group consisting of polyamides, polyesters, polyurethanes, and polyolefins in a layer in contact with the primer layer.

14. The flexible tube for an endoscope according to claim 1,
wherein the resin cover layer has a two-layer structure, and
a ratio of a thickness of an inner layer to a thickness of an outer layer of the two-layer structure changes in a gradient manner in an axial direction of the flexible tube base.

15. The flexible tube for an endoscope according to claim 14, wherein the ratio of the thickness of the inner layer to the thickness of the outer layer is inner layer:outer layer=95:5 to 60:40 at one end of the flexible tube for an endoscope and is inner layer:outer layer=5:95 to 40:60 at the other end.

16. An endoscopic medical device comprising the flexible tube for an endoscope according to claim 1.

17. A method for producing a flexible tube for an endoscope, the method comprising:
a step of forming, on at least an outer periphery of a flexible tube base that contains metal as a constituent material, a primer layer that includes at least one compound represented by general formula (1) or (2); and
a step of forming a resin cover layer, the step including covering, with a resin that includes at least one compound selected from the group consisting of polyamides, polyesters, polyurethanes, and polyolefins, the primer layer formed on the outer periphery of the flexible tube base so as to be in contact with the primer layer:
R¹ₘ-M-(OR²)ₙ₋ₘ General formula (1):
O-[M-(OR²)ₙ₋₁]₂ General formula (2):
where M represents Al, B, Ba, Bi, Ca, Ga, Ge, Hf, In, La, Mg, Nb, P, Sr, Sn, Ta, Ti, V, Y, or Zr;
R¹ represents a hydrogen atom, an alkyl group, a cycloalkyl group, an acyl group, an aryl group, or an unsaturated aliphatic group;
R² represents a hydrogen atom, an alkyl group, a cycloalkyl group, an acyl group, an alkenyl group, an aryl group, a phosphonate group, or —SO₂R^{s} where R^{s} represents a substituent;
m is an integer of 0 to 3; n is a valence of M; and n>m is satisfied.

18. The method for producing a flexible tube for an endoscope according to claim 17,
wherein the resin cover layer has a two-layer structure,
at least an inner layer of the two-layer structure includes at least one compound selected from the group consisting of polyamides, polyesters, polyurethanes, and polyolefins, and
a ratio of a thickness of the inner layer to a thickness of an outer layer of the two-layer structure changes in a gradient manner in an axial direction of the flexible tube base.

19. A method for producing an endoscopic medical device, comprising:
a step of producing a flexible tube for an endoscope by the method for producing a flexible tube for an endoscope according to claim 17; and
a step of incorporating the produced flexible tube for an endoscope into an insertion section of an endoscopic medical device.

20. A method for producing an endoscopic medical device, comprising incorporating the flexible tube for an endoscope according to claim 1 into an insertion section of an endoscopic medical device.
